(19) United States
(12) Patent Application Publication
(10) Pub. No.: US 2004/0204455 A1
(43) Pub. Date: Oct. 14, 2004

(54) Piperidine derivative rennin inhibitors

(76) Inventors: Wayne Livingston Cody, Saline, MI (US); Jeremy John Edmunds, Ypsilanti, MI (US); Daniel Dale Holsworth, Ann Arbor, MI (US); Noel Aaron Powell, Dexter, MI (US)

Correspondence Address: WARNER-LAMBERT COMPANY, 2800 PLYMOUTH RD, ANN ARBOR, MI, 48105, US

(21) Appl. No.: 10/811,200

(22) Filed: Mar. 26, 2004

Related U.S. Application Data
(60) Provisional application No. 60/461,962, filed on Apr. 10, 2003.
(60) Provisional application No. 60/542,279, filed on Feb. 9, 2004.

(51) Int. Cl. A61K 31/445; C07D 211/40

(52) U.S. Cl. 514/317; 546/216

(57) ABSTRACT

Disclosed are piperidine derivatives, their manufacture and use as inhibitors of renin.

The present application claims priority under 35 USC section 119(e) to U.S. Provisional Application Serial No. 60/461,962, filed Apr. 10, 2003 and U.S. Provisional Application Serial No. 60/542,279, filed Feb. 9, 2004.

### FIELD OF THE INVENTION

This invention relates to piperidine derivative useful as inhibitors of renin.

### BACKGROUND OF THE INVENTION

Renin is an endopeptidase (molecular weight about 40,000) produced and secreted by the juxtaglomerular cells of the kidney, which cleaves the naturally-occurring plasma glycoprotein, antiotensinogen. Renin cleaves angiotensinogen, its protein substrate, to split off the hemodynamically-inactive N-terminal decapeptide, angiotensin I, which is converted in the lungs, kidney or other tissue by angiotensin-converting enzyme to the potent pressor octapeptide, angiotensin II. Angiotensin II is known to be a potent pressor substance, i.e., a substance that is capable of inducing a significant increase in blood pressure, and is believed to act by causing the constriction of blood vessels and the release of the sodium-retaining hormone aldosterone from the adrenal gland. Thus, the renin-angiotensinogen system has been implicated as a causative factor in hypertension congestive heart failure, end organ failure, stroke, myocardial infarction, glaucoma and hyperaldosteronism.

Inhibitors of angiotensin I converting enzyme have proven useful in the modulation of the renin-angiotensin system. Consequently, specific inhibitors of the limiting enzymatic step that ultimately regulates angiotensin II production, the action of renin on its substrate, are sought as effective therapeutic agents in the treatment of hypertension, and congestive heart failure.

### SUMMARY OF THE INVENTION

Generally, the present invention relates to piperidine derivative renin inhibitors. One embodiment is a compound of Formula I

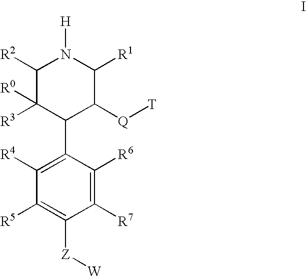

or a pharmaceutically acceptable salt thereof, where

R¹ and R² are independently hydrogen or unsubstituted C₁-C₃ alkyl;

R³ is hydrogen, oxo, or thioxo;

R⁰ is hydrogen or unsubstituted C₁-C₃ alkyl provided that when R³ is oxo or thioxo R⁰ is absent;

R⁴, R⁵, R⁶, and R⁷ are independently hydrogen, halogen, carboxyl, substituted or unsubstituted C₁-C₃ alkoxy, or substituted or unsubstituted C₁-C₃ alkyl;

Q is —NR⁸—(CH₂)₀₋₆—, —NR⁹—C(O)—(CH₂)₀₋₆—, where 1 to 3 nonadjacent methylene units are replaced with O, NR¹⁰, S or a combination thereof;

T is substituted or unsubstituted aryl, substituted or unsubstituted heteroaryl, or substituted or unsubstituted C₁-C₁₂ alkyl;

W is absent, substituted or unsubstituted aryl, or substituted or unsubstituted heteroaryl;

Z is —(CH₂)₀₋₆-cycloalkylene-(CH₂)₀₋₆— where 0 to 6 nonadjacent methylene units are replaced with O, NR¹², S or a combination thereof, —(CH₂)₀₋₆-heterocycloalkylene-(CH₂)₀₋₆— where 0 to 6 nonadjacent methylene units are replaced with O, NR¹², S or a combination thereof, —(CH₂)₀₋₆-arylene-(CH₂)₀₋₆— where 0 to 6 nonadjacent methylene units are replaced with O, NR¹², S or a combination thereof, —(CH₂)₀₋₆-heteroarylene-(CH₂)₀₋₆— where 0 to 6 nonadjacent methylene units are replaced with O, NR¹², S or a combination thereof, —(CH₂)₀₋₆—C(O)—NR¹¹—(CH₂)₀₋₆— where 0 to 6 nonadjacent methylene units are replaced with O, NR¹², S or a combination thereof, —(CH₂)₀₋₆—NR¹¹—C(O)—(CH₂)₀₋₆— where 0 to 6 nonadjacent methylene units are replaced with O, NR¹², S or a combination thereof,

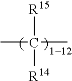

where 1 to 6 nonadjacent

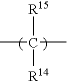

units are replaced with O, NR¹²,

S or a combination thereof, or Z, when W is absent, is hydroxyl, substituted or unsubstituted C₁-C₁₂ alkyl where 1 to 6 nonadjacent methylene units are replaced with O, NR¹⁶, S or a combination thereof, or —(CH₂)_{0.6}—C(O)—NR¹⁶—(CH₂)_{0.5}—CH₃ where 0 to 6 nonadjacent methylene units are replaced with O, NR¹⁶, S or a combination thereof;

R⁸, R⁹ and R¹⁰ are independently hydrogen or substituted or unsubstituted C₁-C₃ alkyl;

R¹ and R¹² are independently substituted or unsubstituted C₁-C₃ alkyl; and

R¹⁴ and R¹⁵ are independently hydrogen, substituted or unsubstituted C₁-C₃ alkoxy, substituted or unsubstituted C₁-C₃ alkyl, unsubstituted C₁-C₁₂ alkyl where 1 to 6 nonadjacent methylene units are replaced with 0, or R¹⁴ and R¹⁵ together with the carbon to which they are attached form a 3- to 6-membered cycloalkylene or heterocycloalkylene ring; and

R¹⁶ is substituted or unsubstituted C₁-C₃ alkyl or hydrogen.

Another embodiment is a compound of Formula IV or V

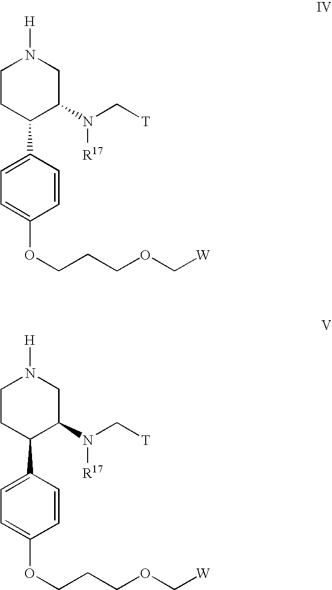

or a pharmaceutically acceptable salt thereof, where

T is substituted or unsubstituted aryl, or substituted or unsubstituted heteroaryl;

W is substituted or unsubstituted aryl, or substituted or unsubstituted heteroaryl; and

R¹⁷ is hydrogen or C₁-C₃ alkyl.

Another embodiment is a pharmaceutical composition comprising a compound of Formula I admixed with a pharmaceutically acceptable carrier, diluent, or excipient.

Another embodiment is a method of inhibiting renin in a mammal comprising administering to the mammal in need thereof an effective amount of a compound of Formula I.

Other embodiments include methods of treating or preventing hypertension, congestive heart failure, stroke, myocardial infarction, glaucoma, or hyperaldosteronism in a mammal comprising administering to the mammal in need thereof an effective amount of a compound of Formula I.

Another embodiment is a method of providing end organ protection in a mammal comprising administering to the mammal in need thereof an effective amount of a compound of Formula I.

Yet another embodiment is a process for preparing a compound of claim 1 including the steps of

a) alkylation of piperidine 1 to afford the intermediate 2 where R²⁰, along with the oxygen to which it is attached, is equivalent to -Z-W as defined above for Formula I;

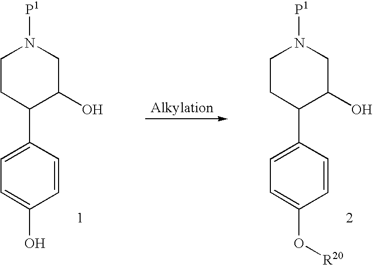

b) oxidation of 2 to afford the piperidinone intermediate 3;

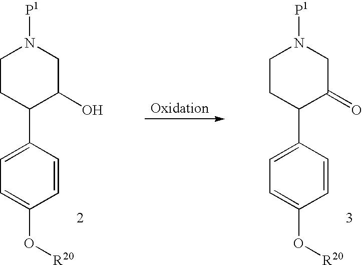

c) contacting 3 with a suitable amine to afford the intermediate 4, where R²¹, along with the nitrogen to which it is attached is equivalent to -Q-T as defined above for Formula I;

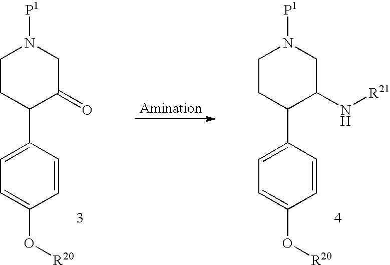

d) deprotection of 4 to afford 5

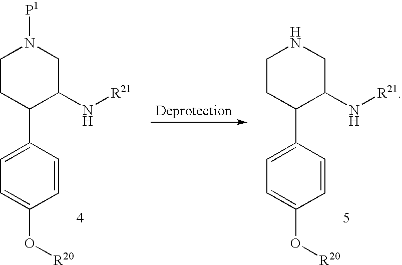

The above summary of the present invention is not intended to describe each disclosed embodiment or every implementation of the present invention. The detailed description which follows more particularly exemplifies these embodiments.

### DETAILED DESCRIPTION OF THE INVENTION

The present invention is believed to be applicable to inhibitors of renin. In particular, the present invention is directed to piperidine derivatives useful as inhibitors of renin. While the present invention is not so limited, an appreciation of various aspects of the invention will be gained through the following discussion and the examples provided below.

Alkyl, alkoxy, etc. denote both straight and branched groups; but reference to an individual radical such as “propyl” embraces only the straight chain radical, a branched chain isomer such as “isopropyl” being specifically referred to.

As used in this specification and the appended claims, the singular forms “a”, “an”, and “the” include plural referents unless the content clearly dictates otherwise. Thus, for example, reference to a composition containing “a compound” includes a mixture of two or more compounds.

The recitation of numerical ranges by endpoints includes all numbers subsumed within that range (e.g. 1 to 5 includes 1, 1.5, 2, 2.75, 3, 3.80, 4, and 5).

The term “halogen” or “halo” as used herein includes chlorine, fluorine, bromine, and iodine.

The term “oxo” as used herein refers to ═O.

The term “thioxo” as used herein refers to ═S.

The term “carboxyl” as used herein refers

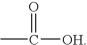

The term “hydroxy” or “hydroxyl” as used herein refers to —OH.

The term “methylene” as used herein refers to —CH₂—.

The term “alkyl” as used herein refers to a monovalent straight or branched hydrocarbon radical having 1 to 12 carbon atoms. Alkyl groups can be unsubstituted or substituted with one or more of the substituents selected from halogen, —OH, —NH₂, or —NHR′, where R′ is unsubstituted C₁-C₃ alkyl. Alkyl groups are assumed to be unsubstituted unless specifically denoted as substituted. Examples of alkyl groups include, but are not limited to, methyl, ethyl, n-propyl, isopropyl, n-butyl, sec-butyl, isobutyl, tert-butyl, n-pentyl, and n-hexyl. Examples of substituted alkyl groups include, but are not limited to, trifluoromethyl, hydroxymethyl, aminomethyl, and methylaminomethyl.

The term “lower” as used herein refers to a group having 1 to 3 carbon atoms. For example “lower alkyl” as used herein refers to a subset of alkyl which means a straight or branched hydrocarbon radical having from 1 to 3 carbon atoms and includes, for example, methyl, ethyl, n-propyl, and isopropyl.

The term “alkylene” as used herein refers to a divalent straight or branched chain hydrocarbon radical having 1 to 12 carbon atoms. Alkylene groups can be unsubstituted or substituted with one or more of the substituents selected from halogen, —OH, —NH₂, or —NHR″, where R″ is unsubstituted C₁-C₃ alkyl. Examples of “alkylene” as used herein include, but are not limited to, methylene, ethylene, propane-1,3-diyl, propane-1,2-diyl, butane-1,4-diyl, pentane-1,5-diyl, and hexane-1,6-diyl.

As used herein, “cycloalkyl” refers to an alicyclic hydrocarbon group having 3 to 8 carbon atoms. Examples of “cycloalkyl” as used herein include, but are not limited to, cyclopropyl, cyclobutyl, cyclopentyl, cyclohexyl, cycloheptyl, and cyclooctyl.

The term “cycloalkylene” as used herein refers to an alicyclic divalent hydrocarbon radical having 3 to 6 carbon atoms. Examples of “cycloalkylene” as used herein include, but are not limited to, cyclopropane-1,1-diyl, cyclopropane-1,2-diyl, cyclobutane-1,2-diyl, cyclopentane-1,1-diyl, cyclopentane-1,3-diyl, cyclohexane-1,1-diyl, cyclohexane-1,2-diyl, cyclohexane-1,3-diyl, cyclohexane-1,4-diyl, cycloheptane-1,4-diyl, and cyclooctane-1,5-diyl.

The term “heterocycloalkyl” as used herein refers to an alicyclic hydrocarbon group having 3 to 6 carbon atoms and containing one to three nonadjacent heteroatomic substitutions independently selected from S, O, and NH. Examples of “heterocycloalkyl” as used herein include, but are not limited to, tetrahydrofuryl, 1,4-dioxyl, 1,3-dioxyl, piperidinyl, pyrrolidinyl, morpholinyl, thiomorpholinyl, tetrahydropyranyl, tetrahydrothiopyranyl, tetrahydrothiophenyl, oxazolidinyl, Isoxazolidinyl, isothiazolidinyl, thiazolidinyl, [1,2]oxathiolanyl, [1,3]oxathiolanyl, [1,2]oxazinanyl, [1,3]oxazinanyl, [1,2]oxathianyl, [1,3]oxathianyl, and [1,4]oxathianyl. For heterocycles containing sulfur, the oxidized sulfur heterocycles containing SO or SO₂ groups are also included. Examples include the sulfoxide and sulfone forms of tetrahydrothiophene.

The term “heterocycloalkylene” as used herein refers to an alicyclic divalent hydrocarbon radical having 3 to 6 carbon atoms and containing one to three nonadjacent heteroatomic substitutions independently selected from S, O, and NH. Examples of “heterocycloalkylene” as used herein include, but are not limited to, tetrahydropyran-4,4-diyl, tetrahydropyran-2,3-diyl, tetrahydropyran-3,4-diyl, tetrahydropyran-2,6-diyl, tetrahydropyran-3,5-diyl, piperidine-4,4-diyl, piperidine-2,3-diyl, piperidine-3,4-diyl, piperidine-2,6-diyl, piperidine-3,5-diyl, tetrahydrothiopyran-4,4-diyl, tetrahydrothiopyran-2,3-diyl, tetrahydrothiopyran-3,4-diyl, tetrahydrothiopyran-2,6-diyl, tetrahydrothiopyran-3,5-diyl, tetrahydrofuran-3,3-diyl, tetrahydrofuran-2,3-diyl, tetrahydrofuran-3,4-diyl, tetrahydrofuran-2,5-diyl, pyrrolidine-3,3-diyl, pyrrolidine-2,3-diyl, pyrrolidine-3,4-diyl, pyrrolidine-2,5-diyl, tetrahydrothiophene-3,3-diyl, tetrahydrothiophene-2,3-diyl, tetrahydrothiophene-3,4-diyl, tetrahydrothiophene-2,5-diyl, morpholine-2,3-diyl, thiomorpholine-2,3-diyl, [1,4]oxathiane-2,3-diyl, oxazolidine-4,5-diyl, [1,3]oxathiolane-4,5-diyl, and thiazolidine-4,5-diyl.

The term “aryl” as used herein means monovalent unsaturated aromatic carbocyclic radicals having a single ring, such as phenyl, or multiple condensed rings, such as naphthyl or anthryl. Aryl groups may be unsubstituted or substituted with 1 to 5 substituents selected from —O(CH₂)₁₋₃CF₃, —NH₂, —OCF₃, —CO₂H, —SO₂(C₁-C₆alkyl), —SO₂NH₂, —SO₂NHR′and —SO₂NR′R″, where R′ and R″ are as defined above, C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl.

Such an aryl ring may be optionally fused to one or more of another heterocycloalkyl ring(s), heteroaryl ring(s), or cycloalkyl rings. Examples of aryl groups include, but are not limited to, anthryl, naphthyl, phenyl, biphenyl, chromanyl, 2-oxo-4a,8a-dihydro-2H-chromenyl 1,2,3,4-tetrahydroquinolinyl, 2-oxo-1,2,3,4-tetrahydroquinolinyl, 3,4-dihydro-2H-benzo[1,4]oxazinyl, 3-oxo-3,4-dihydro-2H-benzo[1,4]oxazinyl, indanyl, 2,3-dihydroindolyl, 1,2,3,4-tetrahydroquinazolinyl, 2-oxo-1,2,3,4-tetrahydroquinazolinyl, 2,3-dihydrobenzoxazolyl, 1,2,3,4-tetrahydronaphthyl, 1,2,3,4-tetrahydroisoquinolinyl, 1,2,3,4-tetrahydroquinoxalinyl, 1,2,3,4-tetrahydro-cinnolinyl, 1,2,3,4-tetrahydro-phthalazinyl, 2,3-dihydroindolyl, 1,2,3,4-tetrahydroindolyl, Specific examples of those aryl groups disclosed immediately above include 2-oxo-1,2,3,4-tetrahydroquinolin-7-yl, 2-oxo-1,2,3,4-tetrahydroquinolin-6-yl, 4-oxo-1,2,3,4-tetrahydroquinolin-7-yl, 4-oxo-1,2,3,4-tetrahydroquinolin-6-yl, 3-oxo-3,4-dihydro-2H-benzo[1,4]oxazin-6-yl, 3-oxo-3,4-dihydro-2H-benzo[1,4]oxazin-7-yl, indan-6-yl, 2-oxo-1,2,3,4-tetrahydroquinazolin-7-yl, 2,3-dihydrobenzoxazol-5-yl, 2-oxo-4a,8a-dihydro-2H-chromen-7-yl, 2,3-dihydroindol-6-yl, 2-oxo-2,3-dihydroindol-6-yl, and 2,3-dihydro-isoindolyl. Examples of substituted 1,2,3,4-tetrahydroquinolinyl include, but are not limited to, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl and 1-(2-acetoxyethyl)-3,4-dihydro-2H-quinolin-7-yl.

Examples of substituted 3,4-dihydro-2H-benzo[1,4]oxazinyl include, but are not limited to, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, and 4-(2-methoxy-2-oxoethyl)-3-oxo-3,4-dihydro-2H-benzo[1,4]oxazin-6-yl.

Examples of substituted naphthyl include, but are not limited to, 6-methoxy-2-naphthyl, 6-hydroxy-2-naphthyl, 7-methoxy-2-naphthyl, 6-methyl-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxy-ethyl)-2-naphthyl, and 7-(2-acetoxy-ethyl)-2-naphthyl.

Examples of substituted phenyl include, but are not limited to, 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, and 4-acetylaminophenyl.

The term “arylene” as used herein refers to divalent unsaturated aromatic carbocyclic radicals having a single ring, such as phenylene, or multiple condensed rings, such as naphthylene or anthrylene. Arylene groups may be unsubstituted or substituted with those substituents enumerated for aryl. Examples of aryl groups include, but are not limited to, phenylene-1,2-diyl, phenylene-1,3-diyl, phenylene-1,4-diyl, naphthalene-2,7-diyl, naphthalene-2,6-diyl, anthracene-1,4-diyl, anthracene-2,6-diyl, and anthracene-2,7-diyl. Examples of substituted arylene groups include, but are not limited to, 2-fluoro-phenylene-1,3-diyl, 2-fluoro-phenylene-1,4-diyl, 2-chloro-phenylene-1,3-diyl, 2-chloro-phenylene-1,4-diyl, 2-methyl-phenylene-1,3-diyl, 2-methyl-phenylene-1,4-diyl, 2-trifluoromethyl-phenylene-1,3-diyl, and 2-trifluoromethyl-phenylene-1,4-diyl.

The term “heteroaryl” as used herein refers to monovalent aromatic cyclic or polycyclic ring systems having from 1 to 4 nonadjacent heteroatoms independently selected from N, O, and S. Heteroaryl groups may be unsubstituted or substituted with one or more groups enumerated for aryl. Examples of heteroaryl include, but are not limited to, thiophenyl, furanyl, pyrrolyl, imidazolyl, pyrazolyl, thiazolyl, isothiazolyl, oxazolyl, isoxazolyl, triazolyl, tetrazolyl, pyridinyl, pyridazinyl, pyrazinyl, pyrimidinyl, quinolinyl, isoquinolinyl, indolyl, quinoxalinyl, benzo[b]thienyl, benzoxazolyl, benzofuryl, benzimidazolyl, benzothiazolyl. Examples of substituted heteroaryl include, but are not limited to, 2-methyl-7-quinolinyl, 2-methyl-6-quinolinyl, 3-methyl-7-quinolinyl, 3-methyl-6-quinolinyl, 2-methoxy-6-quinolinyl, 2-methoxy-7-quinolinyl, 3-methoxy-6-quinolinyl, 3-methoxy-7-quinolinyl, 2-chloro-6-quinolinyl, 2-chloro-7-quinolinyl, 3-chloro-6-quinolinyl, 3-chloro-7-quinolinyl, 2-fluoro-6-quinolinyl, 2-fluoro-7-quinolinyl, 3-fluoro-6-quinolinyl, 3-fluoro-7-quinolinyl, 2-fluoromethyl-6-quinolinyl, 2-fluoromethyl-7-quinolinyl, 3-fluoromethyl-6-quinolinyl, 3-fluoromethyl-7-quinolinyl, 2-(3-hydroxypropyl)-7-quinolinyl, 2-(3-hydroxypropyl)-6-quinolinyl, 2-acetyl-6-quinolinyl, 2-acetyl-7-quinolinyl, 2-(4-thiazolylmethyl)-6-quinolinyl, 2-(4-thiazolylmethyl)-7-quinolinyl, 2-acetamidyl-7-quinolinyl, 2-acetamidyl-6-quinolinyl, 2-(2-acetoxy-ethyl)-7-quinolinyl, 2-(2-acetoxy-ethyl)-6-quinolinyl, 5-benzofuryl, 6-methoxy-2-pyrimidinyl, 5-methoxy-2-pyrimidinyl, 4-methoxy-2-pyrimidinyl, 5-chloro-2-pyridyl, 4-methoxy-2-pyridyl, 5-fluoro-2-pyridyl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, and 1-(2-methoxy-2-oxoethyl)-6-indolyl.

The term “heteroarylene” as used herein refers to divalent aromatic cyclic or polycyclic ring systems having from 1 to 4 heteroatoms independently selected from N, O, and S. Heterorylene groups may be unsubstituted or substituted with those substituents enumerated for aryl. Examples of heteroarylene groups include, but are not limited to, furan-2,5-diyl, thiophene-2,4-diyl, 1,3-thiazole-2,4-diyl, 1,3-thiazole-2,5-diyl, pyridine-2,4-diyl, pyridine-2,3-diyl, pyridine-2,5-diyl, pyrimidine-2,4-diyl, and pyrimidine-2,5-diyl.

The term “alkoxy” as used herein refers to —O-alkyl groups where “alkyl” is defined above.

An “effective amount” is an amount of a compound of the present invention that when administered to a patient ameliorates a symptom of disorders associated with renin activity such as hypertension and congestive heart failure. A therapeutically effective amount of a compound of the present invention can be easily determined by one skilled in the art by administering a quantity of a compound to a patient and observing the result. In addition, those skilled in the art are familiar with identifying patients having disorders associated with renin activity such as hypertension and congestive heart failure.

The term “treating” as used herein refers to the administration of a compound of Formula I, Formula II or pharmaceutically acceptable salts thereof that eliminates, alleviates, inhibits the progression of, or reverses progression of, in part or in whole, any one or more of the pathological hallmarks or symptoms of any one of the diseases and disorders being treated, including, but not limited to, hypertension, congestive heart failure, stroke, myocardial infarction, glaucoma, and hyperaldosteronism.

The term “preventing” as used herein refers to the prophylactic administration of a compound of Formula I, Formula II or pharmaceutically acceptable salts thereof to an asymptomatic patient at risk for the disease or disorder being prevented to inhibit the onset of an associated pathological hallmark or symptom, including, but not limited to, hypertension, congestive heart failure, stroke, myocardial infarction, glaucoma, and hyperaldosteronism. Additionally, once the onset of a pathological hallmark or symptom has begun, preventing means the prevention of further progression or reversal of progression, in part or in whole, of the pathological hallmark or symptom.

The term “pharmaceutically acceptable salts, esters, amides, and prodrugs” as used herein refers to those carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed.

The present invention provides compounds capable of inhibiting renin. Compounds of the present invention are described by Formula I:

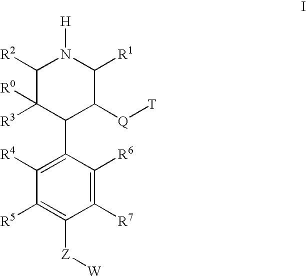

or a pharmaceutically acceptable salt thereof, where

R¹ and R² are independently hydrogen or unsubstituted C₁-C₃ alkyl;

R³ is hydrogen, oxo, or thioxo;

R⁰ is hydrogen or unsubstituted C₁-C₃ alkyl provided that when R³ is oxo or thioxo R⁰ is absent;

R⁴, R⁵, R⁶, and R⁷ are independently hydrogen, halogen, carboxyl, substituted or unsubstituted C₁-C₃ alkoxy, or substituted or unsubstituted C₁-C₃ alkyl;

Q is —NR⁸—(CH₂)₀₋₆—, —NR⁹—C(O)—(CH₂)₀₋₆—, where 1 to 3 nonadjacent methylene units are replaced with O, NR¹⁰, S or a combination thereof;

T is substituted or unsubstituted aryl, substituted or unsubstituted heteroaryl, or substituted or unsubstituted C₁-C₁₂ alkyl;

W is absent, substituted or unsubstituted aryl, or substituted or unsubstituted heteroaryl;

Z is —(CH₂)₀₋₆-cycloalkylene-(CH₂)₀₋₆— where 0 to 6 nonadjacent methylene units are replaced with O, NR¹², S or a combination thereof, —(CH₂)₀₋₆-heterocycloalkylene-(CH₂)₀₋₆— where 0 to 6 nonadjacent methylene units are replaced with O, NR¹², S or a combination thereof, —(CH₂)₀₋₆-arylene-(CH₂)₀₋₆— where 0 to 6 nonadjacent methylene units are replaced with O, NR¹², S or a combination thereof, —(CH₂)₀₋₆-heteroarylene-(CH₂)₀₋₆— where 0 to 6 nonadjacent methylene units are replaced with O, NR¹², S or a combination thereof, —(CH₂)₀₋₆—C(O)—NR¹¹—(CH₂)₀₋₆— where 0 to 6 nonadjacent methylene units are replaced with O, NR¹², S or a combination thereof, —(CH₂)₀₋₆—NR¹¹—C(O)—(CH₂)₀₋₆— where 0 to 6 nonadjacent methylene units are replaced with O, NR¹², S or a combination thereof,

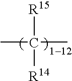

where 1 to 6 nonadjacent

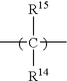

units are replaced with O, NR¹², S or a combination thereof, or

Z, when W is absent, is hydroxyl, substituted or unsubstituted C₁-C₁₂ alkyl where 1 to 6 nonadjacent methylene units are replaced with O, NR¹⁶, S or a combination thereof, or —(CH₂)₀₋₆—C(O)—NR¹⁶—(CH₂)₀₋₅—CH₃ where 0 to 6 nonadjacent methylene units are replaced with O, NR¹⁶, S or a combination thereof;

R⁸, R⁹ and R¹⁰ are independently hydrogen or substituted or unsubstituted C₁-C₃ alkyl;

R¹¹ and R¹² are independently substituted or unsubstituted C₁-C₃ alkyl; and

R¹⁴ and R¹⁵ are independently hydrogen, substituted or unsubstituted C₁-C₃ alkoxy, substituted or unsubstituted C₁-C₃ alkyl, unsubstituted C₁-C₁₂ alkyl where 1 to 6 nonadjacent methylene units are replaced with 0, or R¹⁴ and R¹⁵ together with the carbon to which they are attached form a 3- to 6-membered cycloalkylene or heterocycloalkylene ring; and

R¹⁶ is substituted or unsubstituted C₁-C₃ alkyl or hydrogen.

Examples of compounds of Formula I include those where R¹ and R², are hydrogen and R³ is oxo.

Other examples of compounds of Formula I include those where R⁴, R⁵, R⁶, and R⁷ are independently hydrogen, halogen such as chlorine or fluorine, carboxyl, C₁-C₃ alkoxy such as methoxy, or C₁-C₃ alkyl such as methyl.

Other examples of compounds of Formula I include those where R⁴, R⁶, and R⁷ are hydrogen and R⁵ is chlorine, fluorine, carboxyl, methoxy or methyl

Other examples of compounds of Formula I include those where R⁴, R⁶, and R⁷ are hydrogen and R⁵ is chlorine, fluorine, carboxyl, methoxy or methyl.

Other examples of compounds of Formula I include those where Q is —NR⁸—(CH₂)₀₋₆—, or —NR⁹-C(O)—(CH₂)₀₋₆— where R⁸ and R⁹ are independently unsubstituted C₁-C₃ alkyl.

Additional examples of compounds of Formula I include those where Q is —NH—(CH₂)₀₋₆—, or —NH—C(O)—(CH₂)₀₋₆—.

Additional examples of compounds of Formula I include those where Q is —NH—CH₂—, —NH—CH₂—CH₂—, —NH—CH₂—CH₂—O—CH₂—, or —NH—CH₂—CH₂—O—.

Additional examples of compounds of Formula I include those where T is unsubstituted phenyl, naphthyl such as 2-naphthyl, biphenyl such as biphen-4-yl, 1,2,3,4-tetrahydroquinolinyl such as 1,2,3,4-tetrahydroquinolin-6-yl or 1,2,3,4-tetrahydroquinolin-7-yl, 1,2,3,4-tetrahydro-naphthyl, 1,2,3,4-tetrahydroisoquinolinyl, 1,2,3,4-tetrahydroquinoxalinyl, or 1,2,3,4-tetrahydroindolyl.

Additional examples of compounds of Formula I include those where T is substituted phenyl, naphthyl, biphenyl, 1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl.

Additional examples of compounds of Formula I include those where T is phenyl substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof. For example compounds of Formula I where T is phenyl substituted from 1 to 5 times as stated above include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, and 4-acetylaminophenyl.

Additional examples of compounds of Formula I include those where T is biphenyl substituted from 1 to 9 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁, (C₁-C₆ alkyl)-NR¹⁶—S(O)₂—(C₁-C₆ alkyl)₀₋₁, or HO—(C₁-C₆ alkyl), wherein each R¹⁶ is independently H or C₁-C₆ alkyl or a combination thereof.

Additional examples of compounds of Formula I include those where T is naphthyl, 1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, or 1,2,3,4-tetrahydroindolyl substituted from 1 to 7 times with C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof. Examples of such compounds include 6-methoxy-2-naphthyl, 6-hydroxy-2-naphthyl, 7-methoxy-2-naphthyl, 6-methyl-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxy-ethyl)-2-naphthyl, 7-(2-acetoxy-ethyl)-2-naphthyl, 1-(3-hydroxypropyl)-3,4-dihydro-2H-quinolin-7-yl, 1-acetyl-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, and 1-(2-acetoxy-ethyl)-3,4-dihydro-2H-quinolin-7-yl.

Additional examples of compounds of Formula I include those where T is unsubstituted naphthyl, 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

Further examples of compounds of Formula I include those where T is unsubstituted heteroaryl such as quinolinyl, indolyl, benzofuryl, isoquinolinyl, pyridyl, pyrimidinyl, pyrazinyl, and quinoxalinyl. Examples of compounds of Formula I where T is unsubstituted heteroaryl include 2-quinolinyl, 6-quinolinyl, 7-quinolinyl, 6-isoquinolinyl, 2-pyridyl, 5-benzofuryl, 2-pyrimidinyl, 2-pyrazinyl, and 2-quinoxalinyl.

Further examples of compounds of Formula I include those where T is substituted heteroaryl such as substituted quinolinyl, indolyl, benzofuryl, isoquinolinyl, pyridyl, pyrimidinyl, pyrazinyl, and quinoxalinyl. Examples of compounds of Formula I where T is substituted heteroaryl include quinolinyl, isoquinolinyl, or quinoxalinyl substituted from 1 to 7 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl, or a combination thereof. Other examples include pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶-(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁, (C₁-C₆ alkyl)-NR¹⁶—S(O)₂—(C₁-C₆ alkyl)₀₋₁, or HO—(C₁-C₆ alkyl), wherein each R¹⁶ is independently H or C₁-C₆ alkyl or a combination thereof.

Further examples of compounds of Formula I include those where T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl. Examples of compounds of Formula I where T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl include those where the N-substituent is C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)—NR¹⁶—S(O)₂—(C₁-C₆ alkyl)₀₋₁-, or HO—(C₁-C₆ alkyl), wherein each R¹⁶ is independently H or C₁-C₆ alkyl. Additional N substituents include —(CH₂)₀₋₆—C(O)—O—(CH₂)₀₋₆-L, —(CH₂)₀₋₆—O—C(O)—(CH₂)₀₋₆-L, —(CH₂)₀₋₆—C(O)—NH—(CH₂)₀₋₆-L, —(CH₂)₀₋₆—NH—C(O)—(CH₂)₀₋₆-L, —(CH₂)₀₋₆—NH—S(O)₂—(CH₂)₀₋₆-L, —(CH₂)₀₋₆—S(O)₂—NH—(CH₂)₀₋₆-L, —(CH₂)₀₋₆—NH—C(O)—NH—(CH₂)₀₋₆-L, or —CH₂—(C₁-C₆ alkylene)-L where 1 to 3 nonadjacent methylene units of the alkylene group are replaced with O, NH, S or a combination thereof and where L is aryl, heteroaryl, or heterocycloalkyl.

Further examples of compounds of Formula I include those where Z is

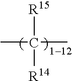

where 1 to 6 nonadjacent

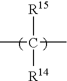

units are replaced with O.

Further examples of compounds of Formula I include those where R¹⁴ and R¹⁵ are hydrogen.

Further examples of compounds of Formula I include those where Z is —(CH₂)₀₋₆—C(O)—NR¹¹—(CH₂)₀₋₆— where 0 to 6 nonadjacent methylene units are replaced with O, NR¹², S or a combination thereof; or —(CH₂)₀₋₆—NR¹¹—(C(O)—CH₂)₀₋₆— where 0 to 6 nonadjacent methylene units are replaced with O, NR¹², S or a combination thereof; and

R¹² is as defined above.

Further examples of compounds of Formula I include those where Z is —O—(CH₂)₂₋₃—O—(CH₂)₁₋₂— such as —O—(CH₂)₃—O—(CH₂)—, —O—(CH₂)₃₋₄—O—, O—(CH₂)₁₋₂—, —(CH₂)—O—(CH₂)₂₋₃—O—(CH₂)₀₋₁—, —C(O)—NR¹⁶—(CH₂)₂—, —C(O)—NR¹⁶—(CH₂)₂—O—, or —O—(CH₂)₃—S—(CH₂)₁—.

Further examples of compounds of Formula I include those where when W is absent, Z is hydroxyl, C₁-C₁₂ alkyl where 1 to 6 nonadjacent methylene units are replaced with O, or —(CH₂)₀₋₆—C(O)—NR¹⁶—(CH₂)₀₋₅—CH₃ where 0 to 6 nonadjacent methylene units are replaced with 0.

Yet further examples of compounds of Formula I include those where W is unsubstituted or substituted phenyl. Examples of compounds of Formula I where W is substituted phenyl include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, and 4-acetylaminophenyl.

Yet further examples of compounds of Formula I include those where W is unsubstituted or substituted heteroaryl. Examples of compounds of Formula I where W is unsubstituted heteroaryl include indolyl such as 1H-Indol-3-yl.

Yet further examples of compounds of Formula I include those where Z is —O—(CH₂)₃—O—CH₂—, and W is 2-methoxyphenyl.

Yet further examples of compounds of Formula I include those where Q is —NH—CH₂— or —NR⁸—CH₂—; T is unsubstituted naphthyl, unsubstituted 4-trifluoromethylphenyl, unsubstituted 1,2,3,4-tetrahydroquinolin-7-yl, 1-(3-hydroxypropyl)-3,4-dihydro-2H-quinolin-7-yl, or 1-(2-acetoxy-ethyl)-3,4-dihydro-2H-quinolin-7-yl; and R⁸ is C₁-C₃ alkyl.

Still further examples of compounds of Formula I include those of Formula II and III:

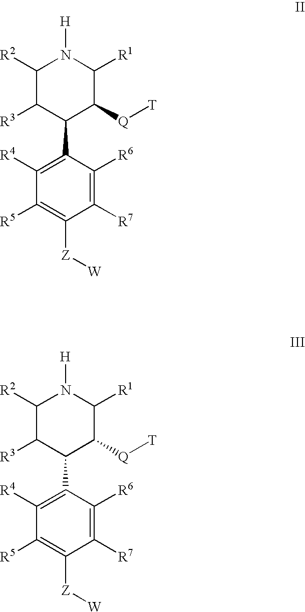

or a pharmaceutically acceptable salt thereof, where

R¹, R², R³, R⁴, R⁵, R⁶, R⁷, R⁸, R⁹, R¹⁰, R¹¹, R¹², R¹⁴, R¹⁵, Q, T, Z, and W are as defined above.

Also within the scope of the invention are compounds of Formula IV and V

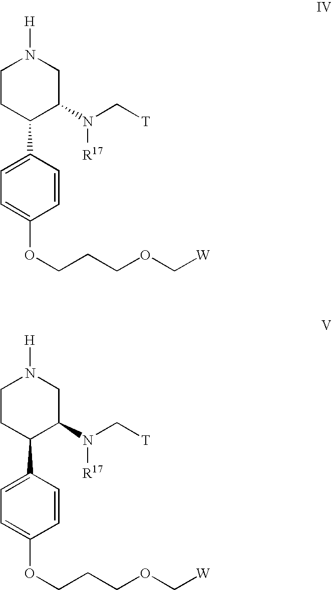

or a pharmaceutically acceptable salt thereof, where

T is substituted or unsubstituted aryl, or substituted or unsubstituted heteroaryl;

W is substituted or unsubstituted aryl, or substituted or unsubstituted heteroaryl; and

R¹⁷ is hydrogen or C₁-C₃ alkyl.

Other examples of compounds of Formula IV and V include those where T is substituted aryl.

Other examples of compounds of Formula IV and V include those where T is phenyl substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶-S(O)₂—(C₁-C₆ alkyl)₀₋₁-, or HO—(C₁-C₆ alkyl), wherein each R¹⁶ is independently H or C₁-C₆ alkyl or a combination thereof. For example compounds of Formula I where T is phenyl substituted from 1 to 5 times as stated above include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, and 4-acetylaminophenyl.

Other examples of compounds of Formula IV and V include those where T is substituted phenyl, naphthyl, biphenyl, 1,2,3,4-tetrahydroquinolinyl, 2-oxo-1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl. Examples of such compounds include 6-methoxy-2-naphthyl, 6-hydroxy-2-naphthyl, 7-methoxy-2-naphthyl, 6-methyl-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxy-ethyl)-2-naphthyl, 7-(2-acetoxy-ethyl)-2-naphthyl, 1-(3-hydroxypropyl)-3,4-dihydro-2H-quinolin-7-yl, 1-acetyl-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, and 1-(2-acetoxy-ethyl)-3,4-dihydro-2H-quinolin-7-yl.

Other examples of compounds of Formula IV and V include those where T is naphthyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 3,4-dihydro-2H-benzo[1,4]oxazinyl, 3-oxo-3,4-dihydro-2H-benzo[1,4]oxazinyl, 2,3-dihydroindolyl, or 1,2,3,4-tetrahydroindolyl substituted from 1 to 7 times with, C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

Other examples of compounds of Formula IV and V include those where T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

Additional examples of compounds of Formula IV and V include those where T is quinolinyl, isoquinolinyl or quinoxalinyl substituted from 1 to 7 times with C₁-C₆ alkyl, halo, C₁-C₆ alkyl wherein 1 to 3 nonadjacent carbons are replaced with O, NR⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶-S(O)₂—(C₁-C₆ alkyl)₀₋₁-, or HO—(C₁-C₆ alkyl), wherein each R¹⁶ is independently H or C₁-C₆ alkyl or a combination thereof.

Further examples of compounds of Formula IV and V include those where T is pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

Yet further examples of compounds of Formula IV and V include those where T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl.

Yet further examples of compounds of Formula IV and V include those where T is C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀-1-, (C₁-C₆ alkyl)-C(O)—(C₁-C₆ alkyl)₀-1-, (C₁-C₆ alkyl)-S(O)₂—NR¹⁶—(C₁-C₆ alkyl)₀₋₁-, (C₁-C₆ alkyl)-NR¹⁶-S(O)₂—(C₁-C₆ alkyl)₀₋₁-, or HO—(C₁-C₆ alkyl), wherein each R¹⁶ is independently H or C₁-C₆ alkyl.

Additional examples of compounds of Formula IV and V include those where W is unsubstituted or substituted phenyl. Examples of compounds of Formula IV where W is substituted phenyl include 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, or 4-acetylaminophenyl.

Additional examples of compounds of Formula IV and V include those where W is 2-methoxyphenyl.

Additional examples of compounds of Formula IV and V include those where T is unsubstituted naphthyl, unsubstituted 4-trifluoromethylphenyl, unsubstituted 1,2,3,4-tetrahydroquinolin-7-yl, 1-(3-hydroxypropyl)-3,4-dihydro-2H-quinolin-7-yl, or 1-(2-acetoxy-ethyl)-3,4-dihydro-2H-quinolin-7-yl and W is 2-methoxyphenyl.

Representative compounds of Formula I include

(4-{4-[3-(2-methoxy-benzyloxy)-propoxy]-phenyl}-piperidin-3-yl)-naphthalen-2-ylmethyl-amine,

(4-{4-[3-(2-methoxy-benzyloxy)-propoxy]-phenyl}-piperidin-3-yl)-(6-methoxy-naphthalen-2-ylmethyl)-amine,

(4-{4-[3-(2-methoxy-benzyloxy)-propoxy]-phenyl}-piperidin-3-yl)-quinolin-7-ylmethyl-amine,

(4-{4-[3-(2-methoxy-benzyloxy)-propoxy]-phenyl}-piperidin-3-yl)-(1,2,3,4-tetrahydro-quinolin-7-ylmethyl)-amine,

(4-{4-[3-(2-methoxy-benzyloxy)-propoxy]-phenyl}-piperidin-3-yl)-methyl-naphthalen-2-ylmethyl-amine,

6-[(4-{4-[3-(2-methoxy-benzyloxy)-propoxy]-phenyl}-piperidin-3-ylamino)-methyl]-naphthalen-2-ol,

benzofuran-5-ylmethyl-(4-{4-[3-(2-methoxy-benzyloxy)-propoxy]-phenyl}-piperidin-3-yl)-amine,

(1H-indol-5-ylmethyl)-(4-{4-[3-(2-methoxy-benzyloxy)-propoxy]-phenyl}-piperidin-3-yl)-amine,

6-[(4-[3-(2-methoxy-benzyloxy)-propoxyl]-phenyl}-piperidin-3-ylamino)-methyl]-naphthalene-1-carboxylic acid methyl ester,

6-[(4-[4-(2-methoxy-benzyloxy)-propoxyl]-phenyl}-piperidin-3-ylamino)-methyl]-naphthalene-1-carboxylic acid,

naphthalene-1-carboxylic acid (4-{4-[3-(2-methoxy-benzyloxy)-propoxy]-phenyl}-piperidin-3-yl)-amide,

6-[(4-(4-[3-(2-methoxy-benyloxy)-propoxy]-phenyl}-piperidin-3-ylamino)-methyl]-naphthalene-2-carboxylic acid methyl ester,

(4-{4-[3-(2-fluoro-benzyloxy)-propoxy]-phenyl}-piperidin-3-yl)-quinolin-7-ylmethyl-amine,

6-[(4-{4-[3-(2-fluoro-benyloxy)-propoxy]-phenyl}-piperidin-3-ylamino)-methyl]-naphthalene-2-carboxylic acid methyl ester,

6-[(4-{4-[3-(2-fluoro-benyloxy)-propoxy]-phenyl}-piperidin-3-ylamino)-methyl]-naphthalene-2-carboxylic acid,

6-[(4-{4-[3-(2-fluoro-benzyloxy)-propoxy]-phenyl}-piperidin-3-ylamino)-methyl]-pyridine-2-carboxylic acid methyl ester,

naphthalene-2-sulfonic acid (4-{4-[3-(2-fluoro-benzyloxy)-propoxy]-phenyl}-piperidin-3-yl)-amide,

(4-{4-[3-(2-fluoro-benzyloxy)-propoxy]-phenyl}-piperidin-3-yl)-(4-fluoro-3-trifluoromethyl-benzyl)-amine,

{3-[(4-{4-[3-(2-fluoro-benzyloxy)-propoxy]-phenyl}-piperidin-3-ylamino)-methyl]-phenoxy}-acetic acid methyl ester,

1-(2-{3-[(4-{4-[3-(2-fluoro-benzyloxy)-propoxy]-phenyl}-piperidin-3-ylamino)-methyl]-phenoxy}-ethyl)-pyrrolidine-2,5-dione,

1-(2-{3-[(4-{4-[3-(2-fluoro-benzyloxy)-propoxy]-phenyl}-piperidin-3-ylamino)-methyl]-phenoxy}-ethyl)-pyrrolidine-2-one,

3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester, and

[1-(2-dimethylamino-ethyl)-1,2,3,4-tetrahydro-quinolin-7-ylmethyl]-(4-{4-[3-(2-methoxybenzyloxy)-propoxy]-phenyl}-piperidin-3-yl)-amine.

Other representative compounds of Formula I include the cis geometric isomers of those compounds listed by name above.

The compounds of Formulae I-V have at least two asymmetric carbon atoms, that being the carbons of the piperidine ring attached to the -Q-T and phenyl moieties, and can exist in the form of optically pure enantiomers racemates, diastereomer mixtures, diastereomeric racemates, or mixtures of diastereomeric racemates. Useful examples of compounds of formulae I-V include those where the relative configuration of the phenyl moiety and the -Q-T moiety is cis.

Processes and novel intermediates for preparing compounds of Formulae I-V are provided as further embodiments of the invention and are illustrated by the following procedures in which the meanings of the generic radicals are as given above unless otherwise qualified. In some cases, protecting groups may have been used to allow synthetic manipulation of one functional group in the presence of other functional groups. It is therefore to be noted that, although not specifically noted in Scheme 1 the appropriate use and choice of protecting groups is well-known by one skilled in the art, and is not limited to the specific examples below. It is also to be understood that such groups not only serve to protect chemically reactive sites, but also to enhance solubility or otherwise change physical properties. A good general reference for protecting group preparation and deprotection is Greene, Theodora, Protective Groups in Organic Synthesis; Wiley: New York, USA, 1991.

Structures encompassed by Formulae I-V can be prepared as described in Scheme 1. The protected hydroxy-piperidine 1 can be prepared according to the method disclosed in Organic Letters, 3, 2317-2320 (2001). The protected hydroxy-piperidine 1, where P¹ is a suitable protecting group such as t-butyloxycarbonyl (BOC) for example, is alkylated to accord the intermediate 2, where R²⁰ along with the oxygen to which it is attached (i.e. the oxygen at the 4-postion of the phenyl ring), is equivalent to -Z-W as is defined above in Formula I. Suitable alkylating agents include halo-R²⁰, such as I—R²⁰ for example. Other examples of suitable alkylating agents include those where R²⁰ is C₁-C₁₂ alkyl, benzyl, 4-trifluoromethylbenzyl, 3,4,5-trifluorobenzyl, 2-naphthylmethyl, 2-methoxybenzyloxy-propyl, 3-methoxybenzyloxypropyl, 4-methoxybenzyloxypropyl, 2-fluorobenzyloxypropyl, benzyloxypropyl, 2-ethoxybenzyloxypropyl, 2-methoxybenzyloxyethyl, 2-methoxyphenoxybutyl, 2-methoxyphenoxypropyl, 3,5-difluorobenzyloxypropyl, 2-chlorobenzyloxypropyl, 3-chlorobenzyloxypropyl, 4-chlorobenzyloxypropyl, 3,4-dichlorobenzyloxypropyl, 4-phenylmethyl, 2-difluoromethoxybenzyl, 3-(2-fluorophenoxy)-benzyl, 2-(3-indolyl)ethyl, and 2-methoxybenzylthiopropyl. The alkylation of 1 can be carried out in an art recognized solvent, such as acetonitrile for example, at about 20° C. to about the reflux temperature of the solvent employed. The intermediate 2 is then oxidized to the corresponding piperidinone 3 using conventional oxidizing reagents, such as pyridinium chlorochromate (PCC), pyridinium dichromate, dipyridine Cr(VI)oxide, MnO₂ or CrO₃, under art recognized conditions. The oxidation of 2 can be carried out in an art recognized solvent, such as dichloromethane for example, at about 0° C. to about 20° C. The piperidinone intermediate 3 is then contacted with an appropriate amine under reductive amination conditions to afford the intermediate 4 where R²¹, along with the nitrogen to which it is attached, is equivalent to -Q-T as is defined above for Formula I. Alternatively, intermediate 3 can be converted to the primary amine and subsequently alkylated to arrive at intermediate 4. Suitable amines can be prepared by those of skill in the art using known reagents and techniques. Suitable amines include, for example, 5-aminomethyl-benzofuran, 5-aminomethyl-indole, 3-aminomehtyl-pyridine, 7-aminomethyl-quinoline, 6-aminomethyl-quinoline, 2-aminomethyl-quinoline, 7-aminomethyl-isoquinoline, 6-aminomethyl-isoquinoline, 2-methylamino-pyridine, 2-methylamino-pyrimidine, 2-methylamino-pyrazine, 2-methylamino-quinoxaline, 7-aminomethyl-1,2,3,4-tetrahydroquinoline, 6-aminomethyl-1,2,3,4-tetrahydroquinoline, 6-aminomethyl-naphthalene, 7-aminomethyl-naphthalene, 6-aminomethyl-naphthalen-2-ol, 7-aminomethyl-naphthalen-2-ol, 7-aminomethyl-3-methoxy-naphthalene, 6-aminomethyl-3-methoxy-naphthalene, 7-aminomethyl-3-methyl-naphthalene, 6-aminomethyl-3-methyl-naphthalene, 7-aminomethyl-3-trifluoromethyl-naphthalene, 6-aminomethyl-3-trifluoromethyl-naphthalene, 7-aminomethyl-3-fluoro-naphthalene, 6-aminomethyl-3-fluoro-naphthalene, 7-aminomethyl-3-chloro-naphthalene, 6-aminomethyl-3-chloro-naphthalene, 7-aminomethyl-3-(2-acetoxy-ethyl)-naphthalene, 6-aminomethyl-3-(2-acetoxy-ethyl)-naphthalene, 3-(7-aminomethyl-3,4-dihydro-2H-quinolin-1-yl)-propan-1-ol, 1-(6-aminomethyl-3,4-dihydro-2H-quinolin-1-yl)-ethanone, (1-thiazol-4-ylmethyl-1,2,3,4-tetrahydro-quinolin-7-yl)-methylamine, 2-(7-aminomethyl-3,4-dihydro-2H-quinolin-1-yl)-acetamide, acetic acid 2-(7-aminomethyl-3,4-dihydro-2H-quinolin-1-yl)-ethyl ester, 2-chloro-benzylamine, 3-chloro-benzylamine, 4-chloro-benzylamine, 2-fluoro-benzylamine, 3-fluoro-benzylamine, 4-fluoro-benzylamine, 2-trifluoromethyl-benzylamine, 3-trifluoromethyl-benzylamine, 4-trifluoromethyl-benzylamine, 2-methyl-benzylamine, 3-methyl-benzylamine, 4-methyl-benzylamine, 2-methoxy-benzylamine, 3-methoxy-benzylamine, 4-methoxy-benzylamine, 3,4-dichloro-benzylamine, 3,5-dichloro-benzylamine, 3,4-difluoro-benzylamine, 3,5-difluoro-benzylamine, 3,4-dimethoxy-benzylamine, 3,5-dimethoxy-benzylamine, 3,4-dimethyl-benzylamine, 3,5-dimethyl-benzylamine, 2-chloro-4-fluoro-benzylamine, 4-fluoro-2-trifluoro-benzylamine, 2-(2-acetoxy-ethyl)-benzylamine, 3-(2-acetoxy-ethyl)-benzylamine, 4-(2-acetoxy-ethyl)-benzylamine, 4-aminomethyl-N,N-dimethyl-benzamide, and 4-acetylamino-benzylamine.

The intermediate 4 is then deprotected to afford the final product 5 which corresponds to compounds of Formula I. Deprotection of intermediate 4 can be accomplished using deprotection methods recognized in the art. For example, the deprotection of intermediate 4 can be accomplished with acetyl chloride in an art recognized solvent such as methanol, at about 0° C. to about the reflux temperature of the solvent employed.

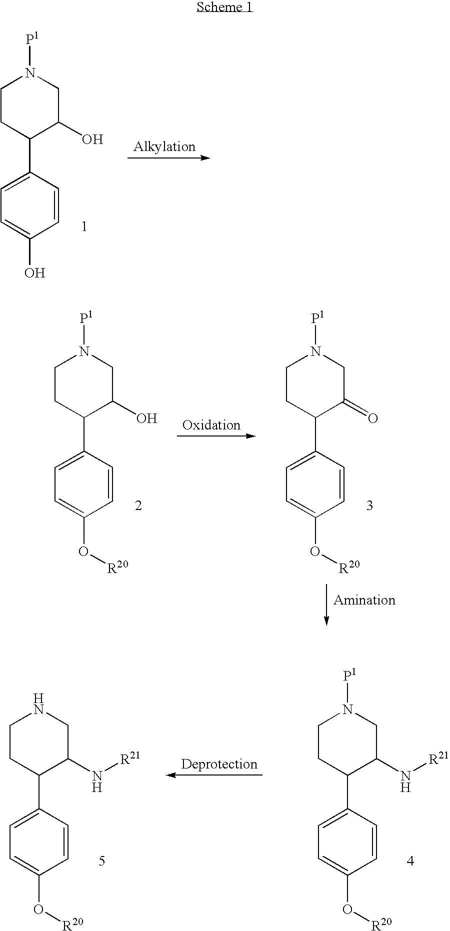

The following non-limiting descriptions also demonstrate methods useful in the synthesis of compounds of Formula I.

Not all compounds of the invention falling into a given class may be compatible with some of the reaction conditions described. Such restrictions are readily apparent to those skilled in the art of organic synthesis, and alternative methods must then be used.

Some of the compounds of Formulae I-V are capable of further forming pharmaceutically acceptable acid-addition and/or base salts. All of these forms are within the scope of the present invention. Thus, pharmaceutically acceptable acid addition salts of the compounds of Formulae I and II include salts derived from nontoxic inorganic acids such as hydrochloric, nitric, phosphoric, sulfuric, hydrobromic, hydriodic, hydrofluoric, phosphorous, and the like, as well as the salts derived from nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, acetate, trifluoroacetate, propionate, caprylate, isobutyrate, oxalate, malonate, succinates suberate, sebacate, fumarate, maleate, mandelate, benzoate, chlorobenzoate, methylbenzoate, dinitrobenzoate, phthalate, benzensoulfonate, toluenesulfonate, phenylacetate, citrate, lactate, maleate, tartrate, methanesulfonate, and the like. Also contemplated are salts of amino acids such as arginate and the like and gluconate, galacturonate (see, for example, Berge S. M. et al., “Pharmaceutical Salts,” Journal of Pharmaceutical Science, 1977;66:1-19).

The acid addition salt of said basic compounds are prepared by contacting the free base form with a sufficient amount of the desired acid to produce the salt in the conventional manner.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge S. M., supra., 1977).

The base addition salts of said acidic compounds are prepared by contacting the free acid form with a sufficient amount of the desired base to produce the salt in the conventional manner.

In some situations, compounds of the invention may exist in isomeric form; for example, as tautomers, enantiomers, or diasteromers. Some compounds may exhibit polymorphism. All tautomers, enantiomers, and diasteromers are incorporated within the definition of the compounds of the invention. It is further to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase) and how to determine activity or cytotoxicity using the standard tests described herein, or using other similar tests which are well known in the art.

Certain of the compounds of the present invention can exist in unsolvated forms as well as solvated forms, including hydrated forms. In general, the solvated forms, including hydrated forms, are equivalent to unsolvated forms and are intended to be encompassed within the scope of the present invention.

The compounds of Formulae I-IV can be formulated as pharmaceutical compositions and administered to a mammalian host, such as a human patient in a variety of forms adapted to the chosen route of administration, i.e., orally or parenterally, by intravenous, intramuscular, or subcutaneous routes. Such pharmaceutical compositions can include a compound of Formula I and a pharmaceutically acceptable carrier and/or adjuvant.

The pharmaceutical compositions may also comprise in addition one or more agents for reducing the risk of a cardiovascular disorder including anti-inflammatory agents, such as alclofenac, algestone acetonide, alpha arnylase, amcinafal, amcinafide, amfenac sodium, amiprilose hydrochloride, anakinra, anirolac, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cortodoxone, deflazacort, desonide, desoximetasone, dexamethasone dipropionate, diclofenac potassium, diclofenac sodium, diflumidone sodium, diflunisal, difluprednate, diftalone, drocinonide, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, ibufenac, ibuprofen, ibuprofen aluminum, ilonidap, indomethacin, indomethacin sodium, indoprofen, indoxole, intrazole, isoflupredone acetate, isoxepac, isoxicam, ketoprofen, lofemizole hydrochloride, lomoxicam, meclofenamate sodium, meclofenamic acid, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salsalate, salycilates, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tolmetin, tolmetin sodium, triclonide, triflumidate, zidometacin, zomepirac sodium; anti-thrombotic and/or fibrinolytic agents, such as plasminogen (to plasmin via interactions of prekallikrein, kininogens, Factors XII, XIIIa, plasminogen proactivator, and tissue plasminogen activator[TPA]) streptokinase, urokinase: anisoylated plasminogen-streptokinase activator complex; pro-urokinase, (Pro-UK); rTPA (alteplase or activase; r denotes recombinant), rPro-UK, abbokinase, eminase, sreptase anagrelide hydrochloride, bivalirudin, dalteparin sodium, danaparoid sodium, dazoxiben hydrochloride, efegatran sulfate, enoxaparin sodium, ifetroban, ifetroban sodium, tinzaparin sodium, retaplase, trifenagrel, warfarin, dextrans; anti-platelet agents, such as clopridogrel, sulfinpyrazone, aspirin; dipyridamole, clofibrate, pyridinol carbamate, PGE, glucagon, antiserotonin drugs, caffeine, theophyllin pentoxifyllin, ticlopidine, anagrelide; lipid reducing agents, such as gemfibrozil, cholystyramine, colestipol, nicotinic acid, probucol lovastatin, fluvastatin, simvastatin, atorvastatin, pravastatin, cirivastatin; and direct thrombin inhibitors, such as hirudin, hirugen, hirulog, agatroban, PPACK, and thrombin aptamers.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Generally, the concentration in a semi-solid or solid composition such as a gel or a powder will be about 0.1-5 wt-%, preferably about 0.5-2.5 wt-%.

Useful dosages of the compounds of Formulae I and II can be determined by comparing their in vitro activity, and in vivo activity in animal models. The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compounds of the present invention can be administered to a patient at dosage levels in the range of about 0.1 to about 2,000 mg per day. For a normal human adult having a body weight of about 70 kilograms, a dosage in the range of about 0.01 to about 10 mg per kilogram of body weight per day is preferable. However, the specific dosage used can vary. For example, the dosage can depended on a numbers of factors including the requirements of the patient, the severity of the condition being treated, and the pharmacological activity of the compound being used. The determination of optimum dosages for a particular patient is well-known to those skilled in the art.

Ideally, the active ingredient should be administered to achieve peak plasma concentrations of the active compound of from about 0.5 to about 75 μM, preferably, about 1 to 50 μM, most preferably, about 0.1 to about 5 μM. This may be achieved, for example, by the intravenous injection of a 0.05 to 5% solution of the active ingredient, optionally in saline, or orally administered as a bolus containing about 10-500 mg of the active ingredient. Desirable blood levels may be maintained by multiple oral dosing, or continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The following examples illustrate the various embodiments of the present invention. Those skilled in the art will recognize many variations that are within the spirit of the present invention and scope of the claims.

### Biological Assays

The ability of a compound of the present invention to inhibit renin is demonstrated using pharmacological models that are well known to the art, for example, using models such as the tests described below.

Determination of Renin IC₅₀ by tGFP FRET Assay

The tGFP FRET (Green Fluorescent Protein Fluorescence Resonance Energy Transfer) assay utilizes a tandem GFP substrate (60 kDa) containing nine amino acid recognition sequences for human renin flanked by two GFP proteins. The assay is used to determine the ability of a compound to act as an inhibitor of renin enzymatic activity by determination of that concentration of test compound that inhibits by 50% (IC₅₀) the ability of renin to cleave the tandem GFP substrate. The IC₅₀ values are determined over an 11-point curve at concentrations of 100 μM to 1 μM. Each compound concentration used to construct the curve was dependent on renin inhibitor potency. For example, subnanomolar IC₅₀ values were determined over an 11-point curve at concentrations of 10 μM to 1 μM. All other IC₅₀ values were determined over an 11-point curve at concentrations of 100 μM to 0.0065 μM. The concentrations were achieved by diluting a 9.1 nM stock of Human recombinant renin in the appropriate amount of buffer containing 50 mM HEPES, 1 mM EDTA, 1% PEG (8000 MW), 1 mM DTT, 0.1% BSA, pH 7.4 to achieve the final concentration of 50.4 μIU. The tGFP substrate stock solution of 43 μM was diluted with the appropriate amount of the above buffer to obtain the final concentration of 650 nM. In addition, 1 μl of the compound is diluted in DSMO to represent an eight-point log scale (5% final). The renin and compound are added to a 384 capacity plate by an automated robot (BIOMEK). The plate is incubated for 60 minutes; upon completion the tGFP substrate is added.

The IC₅₀ is determined by monitoring the increase in absorbance at 432/432 nm excitation, 530/475 nm emission with a cutoff at 515/455 nm, in a fluorometric plate reader. The results of this evaluation are shown in Table 1.

TABLE 1

| Compounds | IC₅₀ μM |
|---|---|
| (4-{4-[3-(2-methoxy-benzyloxy)- | 0.08700 |
| propoxy]-phenyl}-piperidin-3-yl)- | |
| naphthalen-2-ylmethyl-amine | |
| (4-{4-[3-(2-methoxy-benzyloxy)- | 0.07434 |
| propoxy]-phenyl}-piperidin-3-yl)-(6- | |
| methoxy-naphthalen-2-ylmethyl)-amine | |
| (4-{4-[3-(2-methoxy-benzyloxy)- | 0.17500 |
| propoxy]-phenyl}-piperidin-3-yl)- | |
| quinolin-7-ylmethyl-amine | |
| (4-{4-[3-(2-methoxy-benzyloxy)- | 0.22550 |
| propoxy]-phenyl}-piperidin-3-yl)- | |
| (1,2,3,4-tetrahydro-quinolin-7-ylmethyl)- | |
| amine | |
| (4-{4-[3-(2-methoxy-benzyloxy)- | 0.57300 |
| propoxy]-phenyl}-piperidin-3-yl)- | |
| methyl-naphthalen-2-ylmethyl-amine | |
| 6-[(4-{4-[3-(2-methoxy-benzyloxy)- | >1.0 |
| propoxy]-phenyl}-piperidin-3-ylamino)- | |
| methyl]-naphthalen-2-ol | |
| benzofuran-5-ylmethyl-(4-{4-[3-(2- | 0.393 |
| methoxy-benzyloxy)-propoxy]-phenyl}- | |
| piperidin-3-yl)-amine | |
| (1H-indol-5-ylmethyl)-(4-{4-[3-(2- | >1.0 |
| methoxy-benzyloxy)-propoxy]-phenyl}- | |
| pipendin-3-yl)-amine | |
| 6-[(4-[3-(2-methoxy-benzyloxy)- | 0.282 |
| propoxyl]-phenyl}-piperidin-3-ylamino)- | |
| methyl]-naphthalene-1-carboxylic acid | |
| methyl ester | |
| 6-[(4-[4-(2-methoxy-benzyloxy)- | 2.60 |
| propoxyl]-phenyl}-piperidin-3-ylamino)- | |
| methyl]-naphthalene-1-carboxylic acid | |
| naphthalene-1-carboxylic acid (4-{4-[3- | >1.0 |
| (2-methoxy-benzyloxy)-propoxy]- | |
| phenyl}-piperidin-3-yl)-amide | |
| 6-[(4-{4-[3-(2-methoxy-benyloxy)- | 0.329 |
| propoxy]-phenyl}-piperidin-3-ylamino)- | |
| methyl]-naphthalene-2-carboxylic acid | |
| methyl ester | |
| (4-{4-[3-(2-fluoro-benzyloxy)-propoxy]- | 0.400 |
| phenyl}-piperidin-3-yl)-quinolin-7- | |
| ylmethyl-amine | |
| 6-[(4-{4-[3-(2-fluoro-benyloxy)- | 0.877 |
| propoxy]-phenyl}-piperidin-3-ylamino)- | |
| methyl]-naphthalene-2-carboxylic acid | |
| methyl ester | |
| 6-[(4-{4-[3-(2-fluoro-benyloxy)- | >1.0 |
| propoxy]-phenyl}-piperidin-3-ylamino)- | |
| methyl]-naphthalene-2-carboxylic acid | |
| 6-[(4-{4-[3-(2-fluoro-benzyloxy)- | 6.98 |
| propoxy]-phenyl}-piperidin-3-ylamino)- | |
| methyl]-pyridine-2-carboxylic acid | |
| methyl ester | |
| naphthalene-2-sulfonic acid (4-{4-[3-(2- | 0.693 |
| fluoro-benzyloxy)-propoxy]-phenyl}- | |
| piperidin-3-yl)-amide | |
| (4-{4-[3-(2-fluoro-benzyloxy)-propoxy]- | 0.454 |
| phenyl}-piperidin-3-yl)-(4-fluoro-3- | |
| trifluoromethyl-benzyl)-amine | |
| {3-[(4-{4-[3-(2-fluoro-benzyloxy)- | 0.924 |
| propoxy]-phenyl}-piperidin-3-ylamino)- | |
| methyl]-phenoxy}-acetic acid methyl | |
| ester | |
| 1-(2-{3-[(4-{4-[3-(2-fluoro-benzyloxy)- | 1.40 |
| propoxy]-phenyl}-piperidin-3-ylamino)- | |
| methyl]-phenoxy}-ethyl)-pyrrolidine-2,5- | |
| dione | |
| 1-(2-{3-[(4-{4-[3-(2-fluoro-benzyloxy)- | 0.932 |
| propoxy]-phenyl}-piperidin-3-ylamino)- | |
| methyl]-phenoxy}-ethyl)-pyrrolidine-2- | |
| one | |
| 3-[(1-dimethylcarbamoylmethyl-1,2,3, | 0.886 |
| 4-tetrahydro-quinoline-7-carbonyl)- | |
| amino]-4-{4-[3-(2-methoxy-benzyloxy)- | |
| propoxy]-phenyl}-piperidine-1- | |
| carboxylic acid tert-butyl ester | |
| [1-(2-dimethylamino-ethyl)-1,2,3,4- | >1.0 |
| tetrahydro-quinolin-7-ylmethyl]-(4-{4- | |
| [3-(2-methoxybenzyloxy)-propoxy]- | |
| phenyl}-piperidin-3-yl)-amine | |

The foregoing biological tests establish that the compounds of the present invention are potent inhibitors of renin. Accordingly, the compounds of the present invention are useful in pharmaceutical formulations for preventing and treating disorders in which rennin plays a significant pathological role. Such disorders include hypertension and congestive heart failure, end organ protection, stroke, myocardial infarction, glaucoma and hyperaldosteronism.

To further assist in understanding the present invention, the following non-limiting examples of such renin inhibitory compounds are provided. The following examples, of course, should not be construed as specifically limiting the present invention, variations presently known or later developed, which would be within the purview of one skilled in the art and considered to fall within the scope of the present invention as described herein. Preferred synthetic routes for intermediates involved in the synthesis as well as the resulting rennin inhibitory compounds of the present invention follow. All reagents are commercially available (Aldrich Chemical of Milwaukee, Wis.) unless otherwise noted.

### Preparation Methods

Reagents used in the following examples can be prepared using the methods disclosed below in Methods A-M.

Method A: Synthesis of naphthalene-2-yl-methylamine

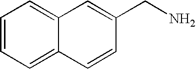

Naphthalene-2-carbonitrile (5.57 g, 36.4 mmoles) was hydrogenated in the presence of Raney Nickel in methanol and aqueous ammonia. The solution was concentrated under reduced pressure to a red semi-solid that was purified on silica gel (EtOAc:MeOH (4:1)), combined and concentrated under reduced pressure to a light pink solid (naphthalene-2-yl-methylamine (4.21 g, 74%).

Method B: Synthesis of C-(6-methoxy-naphthalen-2-yl)-methylamine

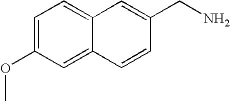

6-Methoxy-naphthalene-2-carbonitrile (5.00 g, 27.0 mmoles) was hydrogenated in the presence of Raney Nickel in methanol and aqueous ammonia. The solution was concentrated under reduced pressure to a semi-solid. The semi-solid was partitioned between ethyl acetate and water (50 mL each), separated, washed with water, brine, dried with magnesium sulfate, filtered and concentrated under reduced pressure to a white solid (C-(6-methoxy-naphthalen-2-yl)-methylamine, 4.13 g, 81%).

Method C: Synthesis of C-quinolin-7-yl-methylamine:

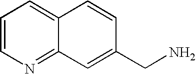

Synthesis of 7-trifluoromethyl-quinoline:

4-Chloro-7-trifluoromethyl-quinoline (19.80 g, 100 mmoles) was hydrogenated in the presence of 5% palladium on carbon in methanol in the presence of triethylamine. The solution was concentrated under reduced pressure, partitioned between ethyl acetate and water (200 mL each), separated, washed with water (2×200 mL), dried with magnesium sulfate, filtered and concentrated under reduced pressure to a yellow solid (7-trifluoromethyl-quinoline, 15.20 g, 90%). H1-NMR was consistent.

Synthesis of quinoline-7-carboxylic acid methyl ester:

7-Trifluoromethyl-quinoline (22.10 g, 112.1 mmols) was dissolved in 30% oleum, heated to 150C for 2 h. The solution was cooled to room temperature and 200 mL of methanol was added slowly and refluxed overnight. The mixture was cooled to room temperature, concentrated under reduced pressure to an oil that was neutralized with saturated sodium carbonate, overlaid with ethyl acetate (100 mL), re-extracted with ethyl acetate (100 mL), dried with magnesium sulfate, filtered and concentrated under reduced pressure to a pink solid (quinoline-7-carboxylic acid methyl ester, 16.10 g, 77%).

Synthesis of quinolin-7-yl methanol:

Quinoline-7-carboxylic acid methyl ester (4.94 g, 26.4 mmols) was dissolved in 70 mL of tetrahydrofuran at −20C under argon. RED-AL (60% in toluene, 12.9 mL, 66 mmols) was added and allowed to stir at −20C for 4 h. After warming to room temperature the reaction was quenched slowly with water, concentrated under reduced pressure, partitioned between ethyl acetate and water (100 mL each), filtered, separated, re-extracted with ethyl acetate, separated, dried with magnesium sulfate and concentrated under reduced pressure. The residue was purified on silica gel in ethyl acetate, appropriate fractions were combined and concentrated under reduced pressure (quinolin-7-yl methanol, 3.42 g, 82%).

Synthesis of 7-bromomethyl-quinoline:

Quinolin-7-yl methanol (3.25 g, 20.4 mmols) was added to a saturated solution of hydrobromic acid in acetic acid (40 mL). The solution was heated to 70C for 4 h, cooled and concentrated under pressure to a light orange oil (7-bromomethyl-quinoline, 6.18 g, 100%).

Synthesis of 7-azidomethyl-quinoline:

7-Bromomethyl-quinoline (2.11 g, 10.0 mmols) was dissolved in 20 mL of DMF and sodium azide (0.975 g, 15.0 mmols) was added and heated to 75C for 16 hours. The solution was cooled, poured into water (100 mL), extracted with EtOAc (2×50 mL), washed with water (2×50 mL), brine (1×50 mL), dried with magnesium sulfate, filtered and concentrated under reduced pressure to a pink oil (7-azidomethy-quinoline, 1.83 g, 99%).

Synthesis of C-quinolin-7-yl-methylamine:

7-Azidomethyl-quinoline (1.76 g, 9.5 mmols) was hydrogenated in the presence of Raney Nickel in methanol at room temperature. The solution was concentrated under reduced pressure to a yellow oil, dissolved in ethyl acetate (50 mL), extracted with 1N hydrochloric acid (3×50 mL), pH adjusted to 10 with 1N sodium hydroxide, extracted with ethyl acetate (3×50 mL), dried with magnesium sulfate, filtered and concentrated under reduced pressure to a white solid (C-quinolin-7-yl-methylamine, 0.811 g, 54%). tlc: Rf=0.00 (EtOAc). H1-NMR and APCI are consistent.

Method D: Synthesis of 6-aminomethyl-naphthalen-2-ol

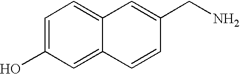

6-Hydroxy-naphthalene-2-carbonitrile was hydrogenated in the presence of Raney Nickel in methanol and aqueous ammonia. The solution was concentrated under reduced pressure to a semi-solid, which was partitioned between ethyl acetate, and water (50 mL each), separated, washed with water (50 mL), brine (50 mL), dried with magnesium sulfate, filtered and concentrated under reduced pressure to a white solid (6-aminomethyl-naphthalen-2-ol, 1.05 g, quantitative).

Method E: Synthesis of C-benzofuran-5-yl-methylamine

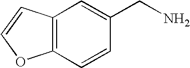

Synthesis of 1-(2,2-diethoxy-ethoxy)-4-methyl-benzene:

p-Cresol (20.0 g, 184.9 mmols), bromoacetaldehyde diethyl acetal (37.2 g, 183.1 mmols) and potassium hydroxide (12.0 g, 183 mmols) were combined in 100 mL of dry DMSO and heated to reflux overnight. The solution turned black, was cooled, poured over ice containing 3.5 grams of sodium hydroxide and diluted to 500 mL with water, extracted with ethyl ether (4×100 mL), combined, washed with 1N sodium hydroxide (1×100 mL), water (4×100 mL), brine (1×100 mL), dried with magnesium sulfate, filtered and concentrated under reduced pressure to a red oil. The was passed through a plug of silica (ethyl acetate:hexanes (1:2)), combined, and concentrated under reduced pressure to a yellow oil (1-(2,2-diethoxy-ethoxy)-4-methyl-benzene, 31.2 g, 76%).

Synthesis of 5-methyl-benzofuran:

(1-(2,2-Diethoxy-ethoxy)-4-methyl-benzene (10.2 g, 45.5 mmols) and polyphosphoric acid (10.2 g) were combined in 200 mL of benzene and brought to reflux for 3.5 hours. Tlc shows disappearance of starting material and a new major spot. The reaction mixture was cooled to room temperature, decanted from the polyphosphoric acid, concentrated under reduced pressure and purified on silica (ethyl acetate:hexanes (1:5)). Fractions were combined and concentrated under reduced pressure to a yellow liquid (5-methyl-benzofuran, 4.61 g, 77%).

Synthesis of 5-bromomethyl-benzofuran:

5-Methyl-benzofuran (4.50 g, 34.0 mmols) was dissolved in carbon tetrachloride (100 mL) and benzoyl peroxide (200 mg) and N-bromosuccinimide (6.06 g, 34.0 mmols) were added. The mixture was refluxed for 30 hours, cooled to room temperature, concentrated under reduced pressure and purified on silica (ethyl acetate:hexanes (1:10)). Appropriate fractions were combined and concentrated under reduced pressure to an orange oil that crystallized overnight which was purified on silica (hexanes), appropriate fractions were combined and concentrated under reduced pressure to a clear oil that crystallized (5-bromomethyl-benzofuran, 2.52 g, 35%).

Synthesis of 5-azidomethyl-benzofuran:

5-Bromomethyl-benzofuran (2.35 g, 11.1 mmols) was dissolved in 20 mL of N,N-dimethylformamide and sodium azide (1.1 g, 17.0 mmols) was added and heated to 75C for 16 h. The solution was cooled, poured into water (100 mL), extracted with ethyl acetate (2×50 mL), washed with water (2×50 mL), brine (1×50 mL), dried with magnesium sulfate, filtered and concentrated under reduced pressure to a yellow oil (5-azidomethyl-benzofuran, 1.907 g, 99%).

Synthesis of C-benzofuran-5-yl-methylamine:

5-Azidomethyl-benzofuran was hydrogenated with Raney Nickel in tetrahydrofuran. The solution was concentrated under reduced pressure to a yellow oil. The oil was dissolved in ethyl acetate (50 mL), extracted with 1N hydrochloric acid (3×50 mL), pH adjusted to 10 with 1N sodium hydroxide, extracted with ethyl acetate (3×50 mL), dried with magnesium sulfate, filtered and concentrated under reduced pressure to a white solid (C-benzofuran-5-yl-methylamine, 0.855 g, 54%).

Method F: Synthesis of C-(1H-indol-5-yl)-methylamine:

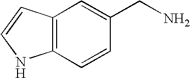

5-Cyanoindole was hydrogenated over Raney Nickel in methanol with aqueous ammonia. The solution was concentrated under reduced pressure to a light yellow solid (C-(1H-indol-5-yl)-methylamine, 5.25 g, quantitative).

Method G: Synthesis of 6-Formyl-naphthalene-2-carboxylic acid methyl ester:

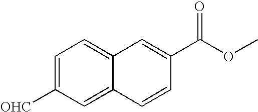

Preparation of 6-Hydroxymethyl-naphthalene-2-carboxylic acid methyl ester: To dimethyl-2,6-naphathlene-dicarboxylate ester (0.5 g) dissoloved in 125 mL of THF at 0° C., was added 1.5M of DIBAL-H (in toluene, 4.5 mL). The reaction was stirred at 0° C. for 30 min, quenched with 5 mL of 2N NaOH, then Na₂CO₃ (sat), filtered and concentrated. The mixture was purified on a silica gel column, eluted with ethyl acetate/hexanes (10 to 65%), to get 6-hydroxymethyl-naphthalene-2-carboxylic acid methyl ester as a white solid (105 mg). 400 MHz ¹H NMR (CDCl₃) δ 7.5-8.6 (m, 6H), 6.85 (d, 1H), 4.13 (d, 2H), 3.95 (s, 3H).

Preparation of 6-Formyl-naphthalene-2-carboxylic acid methyl ester: 6-hydroxymethyl-naphthalene-2-carboxylic acid methyl ester, (0.3 g) in 2 mL of DCM was added, to a mixture of 2.5 mL of pyridine in 25 mL of DCM at 0° C. with CrO₃ (1.4 g). The reaction mixture was stirred at RT for 2 h, filtered through Florisil (200 mL) and purified with a short packed silica gel column, 50% EtOAC/hexanes to give 0.9 g of 6-Formyl-naphthalene-2-carboxylic acid methyl ester as a white solid, 400 MHz ¹H NMR (CDCl₃) δ 10.17(s, 1H), 7.96-8.62 (m, 6H), 6.85 (d, 1H), 3.97 (s, 3H).

Method H: Synthesis of 3-Amino-4-{4-[3-(2-fluoro-benzyloxy)-propoxy]-phenyl}-piperidine-1-carboxylic acid tert-butyl ester:

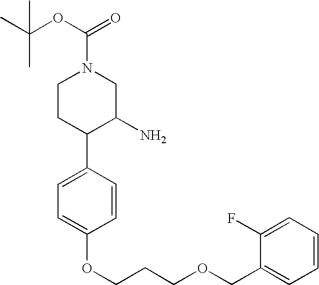

Preparation of 4-{4-[3-(2-Fluoro-benzyloxy)-propoxy]-phenyl}-3-oxo-piperidine-1-carboxylic acid tert-butyl ester: 4-(4-Hydroxy-phenyl)-3-oxo-piperidine-1-carboxylic acid tert-butyl ester (5 g), 6.4 g of 1-(3-Bromo-propoxymethyl)-2-fluoro-benzene, prepared as recited in Method M below, 5 g of potassium carbonate powder and 0.25 g of sodium iodine were combined with 150 mL of 2-propanol, heated to refluxed overnight. The reaction mixture was concentrated, redissolved in 200 mL of ether, filtered and purified on a silica gel column, eluted with 5% to 15% EtOAC/hexanes, to give 3.8 g of 4-{4-[3-(2-Fluoro-benzyloxy)-propoxy]-phenyl}-3-oxo-piperidine-1-carboxylic acid tert-butyl ester, MS m/z 456(M−1).

Preparation of 3-Benzyloxyimino-4-{4-[3-(2-fluoro-benzyloxy)-propoxy]-phenyl}-piperidine1-carboxylic acid tert-butyl ester: 3.8 g of 4-{4-[3-(2-Fluoro-benzyloxy)-propoxy]-phenyl}-3-oxo-piperidine-1-carboxylic acid tert-butyl ester, 1.6 g of O-benzylhydroxyamine hydrochloride and 10 mL of pyridine were combined and stirred at RT overnight. The reaction mixture was concentrated, redissolved in 150 mL of ether and 200 mL of water, washed with water and brine, dried and concentrated, to give 3-Benzyloxyimino-4-{4-[3-(2-fluoro-benzyloxy)-propoxy]-phenyl}-piperidine1-carboxylic acid tert-butyl ester as an oil, (2.5 g). MS m/z 563 (M+1).

Preparation of 3-Amino-4-{4-[3-(2-fluoro-benzyloxy)-propoxy]-phenyl}-piperidine-1-carboxylic acid tert-butyl ester: 3-Benzyloxyimino-4-{4-[3-(2-fluoro-benzyloxy)-propoxy]-phenyl}-piperidine1-carboxylic acid tert-butyl ester (22.4 g) was hydrogenated in MeOH over Raney Nickel (15 g) at 100 psi pressure and RT for 16 h. The reaction mixture was filtered, concentrated and purified on a Al-Oxide column, eluted with 25% EtOAc/hexanes/2% MeOH. 3-Amino-4-{4-[3-(2-fluoro-benzyloxy)-propoxy]-phenyl}-piperidine-1-carboxylic acid tert-butyl ester was isolated as an oil (8.9 g). MS m/z 459 (M+1).

Method I: Synthesis of 6-Formyl-pyridine-2-carboxylic acid methyl ester:

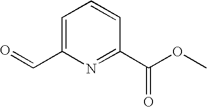

Preparation of 6-Hydroxymethyl-pyridine-2-carboxylic acid methyl ester: One gram of pyridine-2,6-dicarboxylic acid dimethyl ester in 120 mL of MeOH and 300 mg of NaBH₄ were combined and stirred at RT for 3 h. The reaction mixture was concentrated, mixed with 150 mL of EtOAc and 10 mL of NH₄Cl (con) and stirred for 30 min. The organic layer was separated, dried, and purified with a short packed silica, eluted with 20% to 50% EtOAc/hexanes, to afford 6-hydroxymethyl-pyridine-2-carboxylic acid methyl ester as a solid. 450 mg, MS m/z 168 (M+1).

Preparation of 6-Formyl-pyridine-2-carboxylic acid methyl ester: 6-Hydroxymethyl-pyridine-2-carboxylic acid methyl ester (430 mg), 3 g of PCC, and 15 g of Al-oxide in 150 mL of DCM were combined and stirred at RT for 2 h. The reaction mixture was purified through a short packed silica, eluted with 20% EtOAC/hexanes, to afford 6-formyl-pyridine-2-carboxylic acid methyl ester as a white solid. (250 mg). MS m/z 166(M+1)

Method J: Synthesis of 6-Formyl-pyridine-2-carboxylic acid methyl ester:

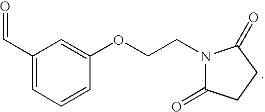

Preparation of Methanesulfonic acid 2-(2,5-dioxo-pyrrolidin-1-yl) ethyl ester: Triethylamine (3.18 g, 31.44 mmol) and methane sulfonylchloride (2.64 g, 23.05 mmol) was added to a solution of N-(2-hydroxyethyl)succinimide (3.0 g, 21 mmol) dissolved in dichloromethane (45 mL). The mixture was stirred at RT for 3 h then diluted with dichloromethane (25 mL) and washed with NH₄Cl (25 mL) and brine (25 mL). The organics were then dried with MgSO₄ and condensed to afford methanesulfonic acid 2-(2,5-dioxo-pyrrolidin-1-yl) ethyl ester. (3.36 g, 72%) MS m/z 222 (M+1).

Preparation of 3-[2-(2,5-Dioxo-pyrrolidin-1-yl)-ethoxy]-benzaldehyde: To a solution of methanesulfonic acid 2-(2,5-dioxo-pyrrolidin-1-yl) ethyl ester (2.0 g, 9.04 mmol) in CH₃CN (40 mL) was added K₂CO₃ (1.5 g, 10.85 mmol) and 3-hydroxy benzaldehyde (1.33 g, 10.85 mmol) dissolved in CH₃CN (10 mL). The mixture was stirred at 80° C. overnight. The reaction mixture was concentrated, diluted with water and CH₂Cl₂, separated and the aqueous layer extracted with CH₂Cl₂ (2×20 mL). The combined organics were dried with MgSO4 and purified by chromatography on silica gel using hexanes and 30% EtOAc to afford 3-[2-(2,5-dioxo-pyrrolidin-1-yl)-ethoxy]-benzaldehyde. (640 mg, 28%). MS m/z 248 (M+1).

Method K: Synthesis of 3-[2-(2-Oxo-pyrrolidin-1-yl)-ethoxy]-benzaldehyde:

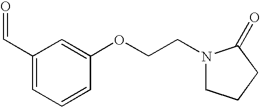

3-[2-(2-Oxo-pyrrolidin-1-yl)-ethoxy]-benzaldehyde was prepared analogously to 3-[2-(2,5-dioxo-pyrrolidin-1-yl)-ethoxy]-benzaldehyde as recited in Method J except that N-(2-hydroxyethyl)-pyrrolidin-2-one was utilized instead of N-(2-hydroxyethyl) succinimide. 18% yield MS m/z 234 (M+1)

Method L: Synthesis of 1-(3-Iodo-propoxymethyl)-2-methoxy-benzene:

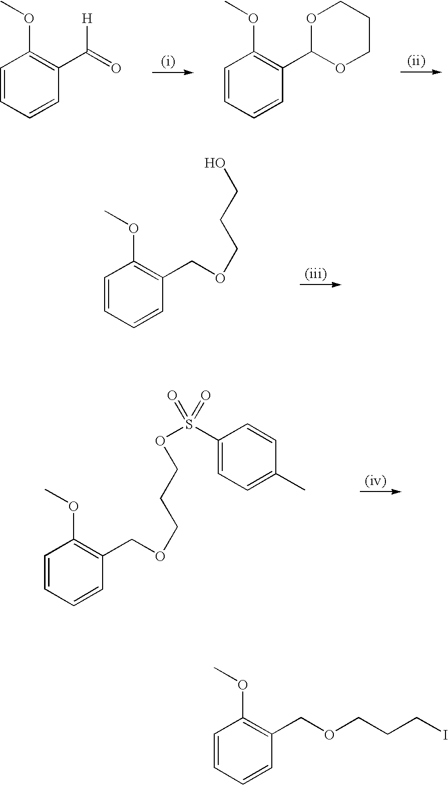

Preparation of 2-(2-Methoxy-phenyl)-[1,3]-dioxane: 2-Methoxy benzaldehyde (30 g, 0.22 mol), propane 1,3-diol (18.44 g, 0.24 mol) and benzene (300 mL) were added to a round bottom flask equipped with a Dean-Stark trap. The reaction mixture was heated to reflux for 5 h and then cooled to room temperature. The mixture was diluted with ethyl acetate (300 mL) and layers separated. The organic layer was washed with water (1×300 mL), 1N HCl (1×100 mL), saturated sodium bicarbonate (1×100 mL) and brine (2×100 mL). The organic layer was dried over magnesium sulfate, filtered and concentrated under reduced pressure to obtain 41 g of a yellow solid. The solid was recrystallized from hexanes to obtain 38.31 g (89%) of 2-(2-methoxy-phenyl)-[1,3]-dioxane (compound ii). MS: m/z 195.1 (M+1).

Preparation of 3-(2-Methoxy-benzyloxy)-propan-1-ol: 2-(2-Methoxy-phenyl)-[1,3]-dioxane (38.3 g, 0.197 mol) was dissolved in toluene (300 mL) under nitrogen. The mixture was cooled to 0° C. and diisobutylaluminum hydride (61.70 g, 0.433 mol) added slowly. Once addition complete, the reaction mixture was allowed to stir 18 h, slowly warming to room temperature. Ethyl acetate (150 mL) was added to quench excess diisobutylaluminum hydride. A solution of 10% Rochelte's salt (800 mL) was added and the mixture stirred for 3 h. Once all salts were dissolved, the layers were separated. The aqueous layer was washed with ethyl acetate (2×400 mL). The organic layer added to the other organic layers. To the aqueous layer was added 10% sodium hydroxide solution (150 mL) to further break up aluminum salts. The aqueous layer was extracted with ethyl acetate (2×150 mL). The organic layers were combined, washed with brine (2×150 mL), dried over anhydrous magnesium sulfate, filtered and concentrated under reduced pressure to afford 37.92 g (98%) of 3-(2-methoxy-benzyloxy)-propan-1-ol (compound iii) as a yellow oil. ¹H NMR (400 MHz, CDCl₃) δ 7.28 (dd, J=7.4, 1.1 Hz, 1H), 7.23 (d, J=9.03 Hz, 1H), 6.91 (t, J=7.4 Hz, 1H), 6.84 (d, J=8.3 Hz, 1H), 4.52 (s, 2H), 3.80 (s, 3H), 3.75 (q, J=5.5 Hz, 2H), 3.68 (t, J=5.6 Hz, 2H), 2.61 (t, J=5.6 Hz, 1H), 1.83 (quintet, J=5.6 Hz, 2H).

Preparation of Toluene-4-sulfonic acid 3-(2-methoxy-benzyloxy)-propyl ester: 3-(2-Methoxy-benzyloxy)-propan-1-ol (37.9 g, 0.193 mol) was dissolved in dichloromethane (300 mL). Dimethylaminopyridine (2.35 g, 0.019 mol), pyridine (16.80 g, 0.212 mol) and tosyl chloride (40.50 g, 0.212 mol) were added at room temperature. The reaction mixture was heated to reflux for 24 h. The mixture was cooled to room temperature and diluted with dichloromethane (400 mL). The layers were separated and the organic layer washed with water (2×200 mL), 1N HCl (2×200 mL), dried over anhydrous magnesium sulfate, filtered and concentrated under reduced pressure to obtain 50 g of solid. The compound was subjected to column chromatography (15-25% ethyl acetate/hexane mixture) to yield 18.28 g (27%) of toluene-4-sulfonic acid 3-(2-methoxy-benzyloxy)-propyl ester (compound iv) as a colorless oil. ¹H NMR (400 MHz, CDCl₃) δ 7.78 (d, J=8.3 Hz, 2H), 7.30 (d, J=8.5 Hz, 2H), 7.26 (dd, J=7.6, 2.0 Hz, 1H), 7.23 (d, J=6.8 Hz, 1H), 6.91 (ddd, J=7.4, 7.4, 1.0 Hz, 1H), 6.85 (d, J=8.3 Hz, 1H), 4.43 (s, 2H), 4.17 (t, J=6.2 Hz, 2H), 3.81 (s, 3H), 3.52 (t, J=6.0 Hz, 2H), 2.40 (s, 3H), 1.94 (quintet, J=6.1 Hz, 2H).

Preparation of 1-(3-Iodo-propoxymethyl)-2-methoxy-benzene: Toluene-4-sulfonic acid 3-(2-methoxy-benzyloxy)-propyl ester (18.22 g, 0.051 mol) was dissolved in acetone (100 mL) under nitrogen. Lithium iodide (10.44 g, 0.077 mol) was added and the mixture heated to reflux for 1 h, cooled to room temperature and filtered through a pad of celite. The celite was washed with acetone and combined with the mother liquor. The organic layer was concentrated under reduced pressure and re-dissolved in dichloromethane. The organic layer was washed with water (2×100 mL), 10% NaS₂O₃ (2×100 mL), brine (2×100 mL), dried over anhydrous magnesium sulfate, filtered and concentrated under reduced pressure to yield 1-(3-iodo-propoxymethyl)-2-methoxy-benzene (17.03 g, 100%) (v) as a yellow oil. ¹H NMR (400 MHz, CDCl₃) δ 7.31 (ddd, J=7.3, 1.0, 0.9 Hz, 1H), 7.23 (ddd, J=8.2, 8.2, 1.6 Hz, 1H), 6.91 (ddd, J=7.4, 7.4, 1.0 Hz, 1H), 6.83 (d, J=8.3 Hz, 1H), 4.52 (s, 2H), 3.80 (s, 3H), 3.54 (t, J=5.7 Hz, 2H), 3.28 (t, J=6.8 Hz, 2H), 2.07 (quintet, J=5.9 Hz, 2H).

Method M: Synthesis of 1-(3-Bromo-propoxymethyl)-2-fluoro-benzene:

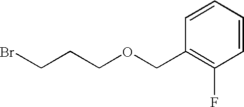

To a solution of 4.7 mL of 3-bromo-1-propanol in 150 mL of THF was added 1.58 g of NaH with stirring. 4.79 mL of 2-fluorobenzylbromide was then added and the reaction mixture stirred overnight at room temperature. The reaction mixture was concentrated, extracted with ether, washed with NaOH and brine, and dried over MgSO4. The reaction mixture was concentrated to afford 1-(3-Bromo-propoxymethyl)-2-fluoro-benzene as an orange oil. 7.89 g, MS nm/z 248 (M+1)

The syntheses described below produce a mixture of the cis stereoisomers.

### EXAMPLES

Example 1

Synthesis of (4-{4-[3-(2-methoxy-benzyloxy)-propoxy]-phenyl}-piperidin-3-yl)-naphthalen-2-ylmethyl-amine

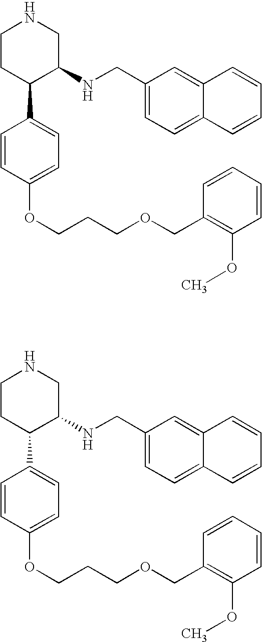

Alkylation of 3-hydroxy-4-(4-hydroxy-phenyl)-piperidine-1-carboxylic acid isopropyl ester: A 250 mL round bottom was charged with 3-hydroxy-4-(4-hydroxy-phenyl)-piperidine-1-carboxylic acid tert-butyl ester (6.58 g, 22.4 mmoles) (prepared as recited in Organic Letters, 3, 2317-2320 (2001)), 1-(3-iodo-propoxymethyl)-2-methoxy-benzene (8.58 g, 28.0 mmoles), and potassium carbonate (4.21 g, 30.5 mmoles) at room temperature in 120 mL of acetonitrile. The solution was brought to reflux overnight. The reaction mixture was cooled, concentrated under reduced pressure, partitioned between ethyl acetate and water (100 mL each), separated, washed with water, brine, separated, dried with magnesium sulfate, filtered and concentrated to an oil (13.58 g). The oil was purified on silica gel (EtOAc:hexanes (1:1)), combined and concentrated under reduced pressure to a clear oil (3-hydroxy-4-{4-[3-(2-methoxy-benzyloxy)-propoxy]-phenyl}-piperidine-1-carboxylic acid tert-butyl ester, 9.94 g, (94%).

Oxidation of 3-Hydroxy-4-{4-[3-(2-methoxy-benzyloxy)-propoxy]-phenyl}-piperidine-1-carboxylic acid tert-butyl ester: 3-Hydroxy-4-(4-[3-(2-methoxy-benzyloxy)-propoxy]-phenyl}-piperidine-1-carboxylic acid tert-butyl ester (9.94 g, 21.1 mmoles), pyridinium chlorochromate (PCC, 6.8 g, 32 mmoles), celite (6.8 g) and crushed 4A molecular sieves (6.8 g) were combined in 100 mL of dichloromethane at room temperature. The solution was allowed to stir overnight and tlc (EtOAc:hexanes (3:2)) and APCI indicated that the reaction was incomplete (˜50-60%). An additional 0.75 equivalents of PCC, celite, and sieves were added. The solution was filtered through celite, washed with ethyl ether, combined and concentrated under reduced pressure to an oil. This oil was chromatographed on silica (EtOAc:hexanes (1:2)), appropriate fractions were combined and concentrated under reduced pressure (4-{4-[3-(2-Methoxy-benzyloxy)-propoxy]-phenyl}-3-oxo-piperidine-1-carboxylic acid tert-butyl ester, 3.67 g, (37.1%).

Reductive amination of 4-{4-[3-(2-Methoxy-benzyloxy)-propoxy]-phenyl}-3-oxo-piperidine-1-carboxylic acid tert-butyl ester: 4-{4-[3-(2-Methoxy-benzyloxy)-propoxy]-phenyl}-3-oxo-piperidine-1-carboxylic acid tert-butyl ester (1.85 g, 3.94 mmoles), naphthalen-2-yl-methylamine (0.93 g, 5.9 mmoles and acetic acid (0.225 mL, 3.94 mmoles), prepared as in Method A, were combined in 20 mL of dichloromethane at room temperature under argon. After 30 minutes sodium triacetoxyborohydride (1.3 g, 5.9 mmoles) was added and the solution was allowed to stir overnight. The reaction was quenched with saturated sodium bicarbonate, partitioned between ethyl acetate and H₂O (25 mL each), separated, dried with magnesium sulfate, filtered and concentrated under reduced pressure to a yellow oil (2.79 g). Appropriate fractions were combined and concentrated under reduced pressure to a yellow solid (4-{4-[3-(2-methoxy-benzyloxy)-propoxy]-phenyl}-3-[(naphthalen-2-ylmethyl)-amino]-piperidine-1-carboxylic acid tert-butyl ester, 1.36 g, 56.5%).

Deprotection of 4-{4-[3-(2-methoxy-benzyloxy)-propoxy]-phenyl}-3-[(naphthalen-2-ylmethyl)-amino]-piperidine-1-carboxylic acid tert-butyl ester: Two hundred mg of 4-{4-[3-(2-methoxy-benzyloxy)-propoxy]-phenyl}-3-[(naphthalen-2-ylmethyl)-amino]-piperidine-1-carboxylic acid tert-butyl ester was dissolved in 5 mL of methanol at 0° C. under argon. Acetyl chloride (233 uL) was added and allowed to stir overnight while warming to room temperature. The reaction was complete by RP-HPLC and purified by RP-HPLC. Fractions were concentrated to a white powder. The white powder was dissolved in methanol (2 mL) and water was added to the precipitation point, saturated sodium bicarbonate was added and a precipitate formed that was absorbed to C18, washed with water, and eluted with tetrahydrofuran. The effluent was combined with water and lyophilized to afford ((4-{4-[3-(2-methoxy-benzyloxy)-propoxy]-phenyl}-piperidin-3-yl)-naphthalen-2-ylmethyl-amine, 52.8 mg, 31.6%). MS: m/z 511.2 (M+1).

### Example 2

Synthesis of (4-{4-[3-(2-methoxy-benzyloxy)-propoxy]-phenyl}-piperidin-3-yl)-(6-methoxy-naphthalen-2-ylmethyl)-amine

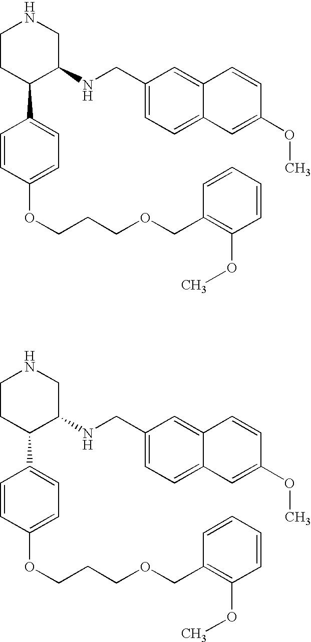

The title compound was prepared as recited in Example 1 utilizing C-(6-methoxy-naphthalen-2-yl)-methylamine instead of C-naphthalen-2-yl-methylamine, prepared as in Method B, in the reductive amination step. M+1=541.2.

### Example 3

Synthesis of (4-{4-[3-(2-methoxy-benzyloxy)-propoxy]-phenyl}-piperidin-3-yl)-quinolin-7-ylmethyl-amine

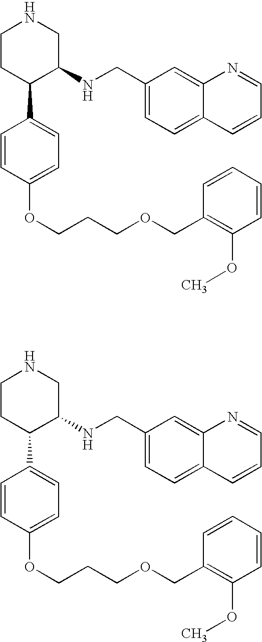

The title compound was prepared as recited in Example 1 utilizing C-quinolin-7-yl-methylamine, prepared as in Method C, instead of C-naphthalen-2-yl-methylamine in the reductive amination step. MS: m/z 512.2 (M+1).

### Example 4

Synthesis of (4-{4-[3-(2-methoxy-benzyloxy)-propoxy]-phenyl}-piperidin-3-yl)-(1,2,3,4-tetrahydro-quinolin-7-ylmethyl)-amine

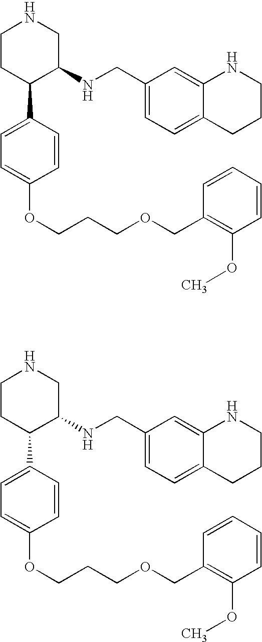

4-{4-[3-(2-Methoxy-benzyloxy)-propoxy]-phenyl}-3-[(quinolin-7-ylmethyl)-amino]-piperidine-1-carboxylic acid tert-butyl ester, prepared in Example 3, (0.394 g, 0.64 mmoles) and nickel(II) chloride hexahydrate (0.077 g, 0.32 mmoles) were dissolved in 5 mL of methanol at 0° C. under argon. After 30 min. sodium borohydride (0.100 g, 3.0 mmoles) was added in two portions and allowed to stir at 0° C. for 4 hours at room temperature. The solution was recooled to 0° C. and another 0.5 eq. of nickel(II) chloride hexahydrate and sodium borohydride was added and allowed to stir overnight while warming to room temperature. The solution was poured into a solution of saturated ammonium chloride (20 mL) and EtOAc (40 mL) and stirred vigorously for 15 min., separated, extracted with EtOAc (2×25 mL), dried with magnesium sulfate, filtered and concentrated under reduced pressure to afford ((4-{4-[3-(2-methoxy-benzyloxy)-propoxy]-phenyl}-3-[(1,2,3,4-tetrahydro-quinolin-7-ylmethyl)-amino]-piperidine-1-carboxylic acid tert-butyl ester as a clear oil, 0.3985 g, (100%). The remaining Boc protecting group was removed as in Example 1 to yield the title compound. MS: m/z 516.3 (M+1).

### Example 5

Synthesis of (4-{4-[3-(2-methoxy-benzyloxy)-propoxy]-phenyl}-piperidin-3-yl)-methyl-naphthalen-2-ylmethyl-amine

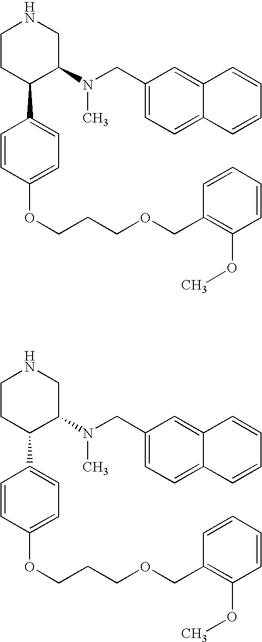

4-{4-[3-(2-Methoxy-benzyloxy)-propoxy]-phenyl}-3-[(naphthalen-2-ylmethyl)-amino]-piperidine-1-carboxylic acid tert-butyl ester (0.295 g, 0.483 mmoles), prepared in Example 1, was dissolved in 4 mL of dichloromethane and 190 uL (2.42 mmoles) of formaldehyde was added at room temperature with stirring. Two drops of acetic acid were added and the solution turned yellow. After approximately 30 min. sodium triacetoxyborohydride (0.15 g, 0.72 mmoles) was added and allowed to stir for two hours. The solution was diluted with dichloromethane (25 mL), washed with water (1×25 mL), saturated sodium bicarbonate (1×25 mL), brine (1×25 mL), dried with magnesium sulfate, filtered and concentrated under reduced pressure to afford ((4-{4-[3-(2-methoxy-benzyloxy)-propoxy]-phenyl}-3-(methyl-naphthalen-2-ylmethyl-amino)-piperidine-1-carboxylic acid tert-butyl ester as a yellow oil, 0.290 g, (96%). The remaining Boc protecting group was removed as in Example 1 to yield the title compound. MS: m/z 525.3 (M+1).

### Example 6

Synthesis of 6-[(4-{4-[3-(2-methoxy-benzyloxy)-propoxy]-phenyl}-piperidin-3-ylamino)-methyl]-naphthalen-2-ol

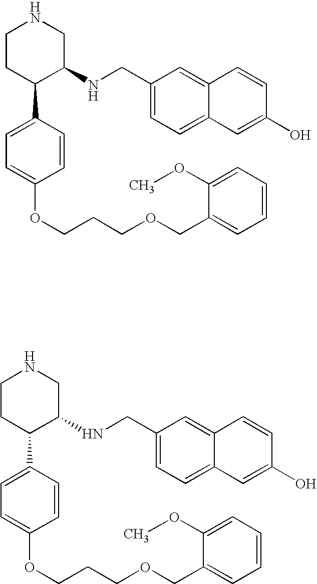

The title compound was prepared as recited in Example 1 utilizing 6-aminomethyl-naphthalen-2-ol, prepared as in Method D, instead of C-naphthalen-2-yl-methylamine in the reductive amination step. MS: m/z 527.2 (M+1).

### Example 7

Synthesis of benzofuran-5-ylmethyl-(4-{4-[3-(2-methoxy-benzyloxy)-propoxy]-phenyl}-piperidin-3-yl)-amine

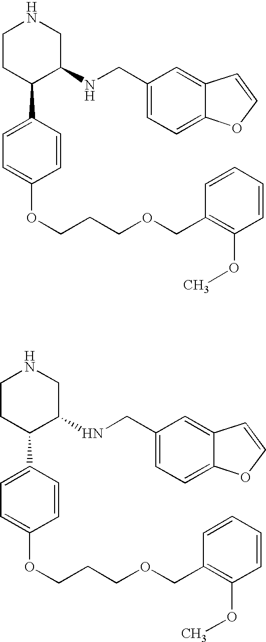

The title compound was prepared as recited in Example 1 utilizing C-benzofuran-5-yl-methylamine, prepared as in Method E, instead of C-naphthalen-2-yl-methylamine in the reductive amination step.

### Example 8

Synthesis of (1H-indol-5-ylmethyl)-(4-{4-[3-(2-methoxy-benzyloxy)-propoxy]-phenyl}-piperidin-3-yl)-amine

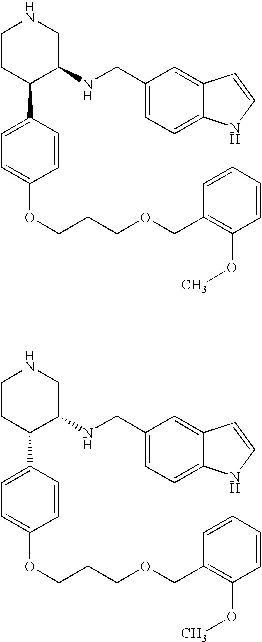

The title compound was prepared as recited in Example 1 utilizing C-(1H-Indol-5-yl)-methylamine instead, prepared as in Method E, of C-naphthalen-2-yl-methylamine in the reductive amination step.

### Example 9

Synthesis of 6-[(4-[3-(2-methoxy-benzyloxy)-propoxyl]-phenyl}-piperidin-3-ylamino)-methyl]-naphthalene-1-carboxylic acid methyl ester

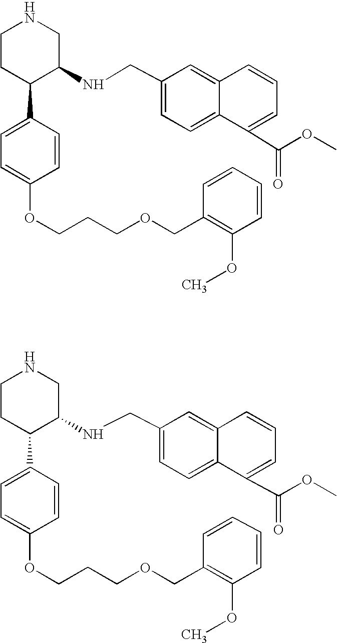

Reductive amination of 4-{4-[3-(2-methoxy-benzyloxy)-propoxyl]-phenyl}-3-oxo-piperidine-carboxylic acid tert-butyl ester: 4-{4-[3-(2-Methoxy-benzyloxy)-propoxyl]-phenyl}-3-oxo-piperidine-carboxylic acid tert-butyl ester (2.95 g, 6.28 mmoles) and O-benzylhydroxylamine hydrochloride (1.10 g, 6.90 mmoles) were combined in 15 mL pyridine at room temperature under argon and allowed to stir overnight. The solution was concentrated and filtered to yield 4-{4-[3-(2-methoxy-benzyloxy)-propoxyl]-phenyl}-piperidine-3-one O-benzyl oxime (2.983 g, 82.6%). MS: m/z 575.2 (M+1).

Hydrogenation of 4-{4-[3-(2-methoxy-benzyloxy)-propoxyl]-phenyl}-piperidine-3-one O-benzyl oxime: 4-{4-[3-(2-Methoxy-benzyloxy)-propoxyl]-phenyl}-piperidine-3-one O-benzyl oxime (2.88 g, 5.018 mmoles) and 5.0 grams of Raney Nickel were dissolved in 100 mL of tetrahydrofuran and placed under a hydrogen atmosphere for 17.5 hours. The solution was concentrated under reduced pressure to a clear oil (2.901 g), which was chromatographed on silica gel (dichloromethane:methanol, 95:5), appropriate fractions were combined and concentrated under reduced pressure to yield 3-amino-4-{4-[3-(2-methoxy-benzyloxy)-propoxy]-phenyl)-piperidine 1-carboxylic acid tert-butyl ester (0.795 g, 37%). MS: m/z 471.3 (M+1).

Alkylation of 3-amino-4-{4-[3-(2-methoxy-benzyloxy)-propoxy]-phenyl)-piperidine 1-carboxylic acid tert-butyl ester: 3-Amino-4-{4-[3-(2-methoxy-benzyloxy)-propoxy]-phenyl)-piperidine 1-carboxylic acid tert-butyl ester (0.312 g, 0.663 mmoles) was dissolved in 5 mL of dry tetrahydrofuran at room temperature under argon. Sequentially, 6-bromomethyl-naphthalene-1-carboxylic acid methyl ester (0.280 g, 1.00 mmoles) and triethylamine (0.215 g, 2.12 mmoles) were added and the solution was brought to reflux overnight. The solution was purified directly on silica (10% ethyl acetate:hexanes to 70% ethyl acetate:hexanes over 45 min.), appropriate fractions were combined and concentrated under reduced pressure to yield 4-{4-[-(2-methoxy-benzyloxy)-propoxyl]-phenyl}-3-[(5-methoxycarbonyl-naphthalen-2-ylmethyl)-amino]-piperidine 1-carboxylic acid tert-butyl ester (0.225 g, 42%). MH: m/z 669.4 (M+1).

Deprotection of 4-{4-[-(2-methoxy-benzyloxy)-propoxyl]-phenyl}-3-[(5-methoxycarbonyl-naphthalen-2-ylmethyl)-amino]-piperidine 1-carboxylic acid tert-butyl ester: 4-{4-[-(2-methoxy-benzyloxy)-propoxyl]-phenyl}-3-[(5-methoxycarbonyl-naphthalen-2-ylmethyl)-amino]-piperidine 1-carboxylic acid tert-butyl ester (0.225 g, 0.336 mmoles) was dissolved in 5 mL of dry methanol at OC under argon. After 30 minutes acetyl chloride (0.264 g, 3.364 mmoles) was added and allowed to stir overnight while warming to room temperature. The solution was purified directly on a Vydac 218TP1022 column (A:0.1% TFA/H₂O, B:0.1% TFA/AcCN, Gradient 10-70% B over 120 min.). Appropriate fractions were combined and lyophilized to a white powder. The powder was dissolved in methanol, excess saturated sodium bicarbonate was added, absorbed to C18, eluted with methanol, diluted with water and lyophilized to yield. 6-[(4-[3-(2-methoxy-benzyloxy)-propoxyl]-phenyl}-piperidin-3-ylamino)-methyl]-naphthalene-1-carboxylic acid methyl ester (67 mg, 35.0%). MH: m/z 569.3 (M+1).

### Example 10

Synthesis of 6-[(4-[4-(2-methoxy-benzyloxy)-propoxyl]-phenyl}-piperidin-3-ylamino)-methyl]-naphthalene-1-carboxylic acid

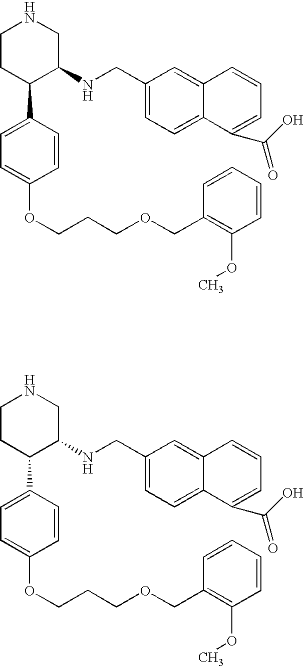

Hydrolysis of 6-[(4-[3-(2-methoxy-benzyloxy)-propoxyl]-phenyl}-piperidin-3-ylamino)-methyl]-naphthalene-1-carboxylic acid methyl ester: 6-[(4-[3-(2-methoxy-benzyloxy)-propoxyl]-phenyl}-piperidin-3-ylamino)-methyl]-naphthalene-1-carboxylic acid methyl ester, prepared as in Example 9, was dissolved in 4 mL of methanol:water (3:1) at room temperature with stirring. Lithium hydroxide was added and the solution was allowed to stir overnight. The reaction mixture was concentrated to remove the methanol, absorbed to C18, washed with water, eluted with tetrahydrofuran, concentrated and lyophilized to yield 6-[(4-[4-(2-methoxy-benzyloxy)-propoxyl]-phenyl}-piperidin-3-ylamino)-methyl]-naphthalene-1-carboxylic acid (15.2 mg, 13%). MH: m/z 555.3 (M+1).

### Example 11

Synthesis of naphthalene-1-carboxylic acid (4-{4-[3-(2-methoxy-benzyloxy)-propoxy]-phenyl}-piperidin-3-yl)-amide

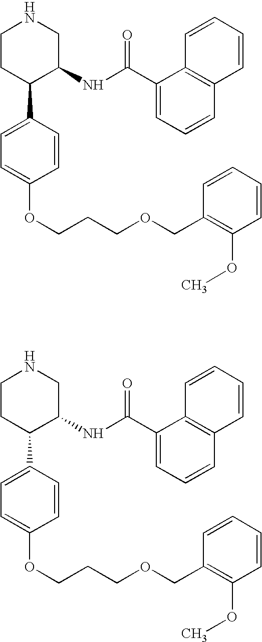

The title compound was prepared from 3-Amino-4-{4-[3-(2-methoxy-benzyloxy)-propoxy]-phenyl)-piperidine 1-carboxylic acid tert-butyl ester as recited in Example 9 utilizing naphthalene-1-carbonyl chloride instead of 6-bromomethyl-naphthalene-1-carboxylic acid tert-butyl ester in the alkylation step. ME: m/z 625.3 (M+1). Deprotection of the resulting 4-{4-[3-(2-methoxy-benzyloxy)-propoxy]-phenyl}-3-[(naphthalene-1-carbonyl)-amino]-piperdine-carboxylic acid tert-butyl ester according to the method in Example 9 afforded the title compound. (36 mg, 43%). MH: m/z 525.2 (M+1).

### Example 12

Synthesis of 6-[(4-{4-[3-(2-Methoxy-benyloxy)-propoxy]-phenyl}-piperidin-3-ylamino)-methyl]-naphthalene-2-carboxylic acid methyl ester

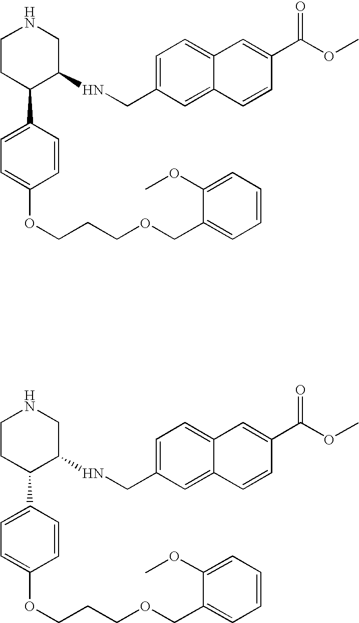

Preparation of 4-{4-[3-(2-Methoxy-benzyloxy)-propoxy]-phenyl}-3-[(6-methoxycarbonyl-naphthalen-2-ylmethyl)-amino]-piperidine-1-carboxylic acid tert-butyl ester: 3-Amino-4-{4-[3-(2-methoxy-benzyloxy)-propoxy]-phenyl}-piperidine-1-carboxylic acid tert-butyl ester, prepared as recited in Example 9 (150 mg), 6-formyl-naphthalene-2-carboxylic acid methyl ester prepared according to method G (150 mg), were combined with AcOH (20 mg) in 5 mL of DCM, at room temp, stirred and NaBH(OAc)₃ (150 mg) was added and stirred overnight. 2 mL of NaHCO3(Sat) and 5 g of NaHCO3 was sequentially added with stirring in between additions, filtered and concentrated. The reaction mixture was purified with a short packed silica gel column, eluted with 5% to 10% EtOAc/hexanes, to get 4-{4-[3-(2-Methoxy-benzyloxy)-propoxy]-phenyl}-3-[(6-methoxycarbonyl-naphthalen-2-ylmethyl)-amino]-piperidine-1-carboxylic acid tert-butyl ester as a white solid (100 mg), MS m/z 669(M+1).

Preparation of 6-[(4-{4-[3-(2-Methoxy-benyloxy)-propoxy]-phenyl}-piperidin-3-ylamino)-methyl]-naphthalene-2-carboxylic acid methyl ester: 100 mg of 4-{4-[3-(2-Methoxy-benzyloxy)-propoxy]-phenyl}-3-[(6-methoxycarbonyl-naphthalen-2-ylmethyl)-amino]-piperidine-1-carboxylic acid tert-butyl ester and 0.125 g of acetyl chloride in 2 mL of DCM and 10 mL of MeOH, were combined and stirred for three days. 2 mL of NaHCO₃(con) and 10 mL of EtOAc were added and stirred for 30 min. The reaction mixture was filtered through a short packed Al-Oxide (15 g), eluted with 1% to 20% MeOH/hexane, to give 45 mg of Preparation of 6-[(4-{4-[3-(2-Methoxy-benyloxy)-propoxy]-phenyl}-piperidin-3-ylamino)-methyl]-naphthalene-2-carboxylic acid methyl ester, MS m/z 569 (M+1).

### Example 13

Synthesis of (4-{4-[3-(2-Fluoro-benzyloxy)-propoxy]-phenyl}-piperidin-3-yl)-quinolin-7-ylmethyl-amine

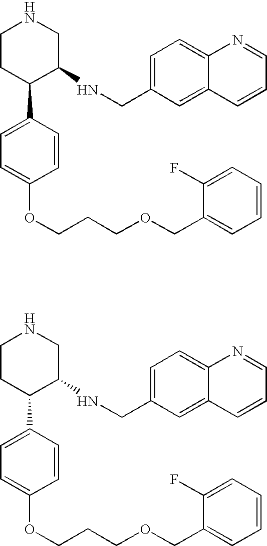

Preparation of 4-{4-[3-(2-Fluoro-benzyloxy)-propoxy]-phenyl}-3-[(quinolin-7-ylmethyl)-amino]-piperidine-1-carboxylic acid tert-butyl ester:

3-Amino-4-{4-[3-(2-fluoro-benzyloxy)-propoxy]-phenyl}-piperidine-1-carboxylic acid tert-butyl ester, prepared as in Example H, (300 mg), quinoline-7-carbaldehyde (150 mg), prepared by reacting 7-methylquinoline and selenium dioxide at 160° C., and AcOH(20 mg) in 5 mL of DCM were combined and stirred at RT for 45 min. NaBH(OAc)₃ (150 mg) was then added and the reaction stirred at RT overnight. 2 mL of NaHCO₃(Sat) and 5 g of NaHCO₃ were then added sequentially with stirring. The reaction mixture was filtered, concentrated and purified with a short packed silica gel column, eluted with 5% to 35% EtOAc/hexanes, to get the title compound as a white solid (155 mg), MS m/z 600 (M+1).

Deprotection of 4-{4-[3-(2-Fluoro-benzyloxy)-propoxy]-phenyl}-3-[(quinolin-7-ylmethyl)-amino]-piperidine-1-carboxylic acid tert-butyl ester: 4-{4-[3-(2-Fluoro-benzyloxy)-propoxy]-phenyl}-3-[(quinolin-7-ylmethyl)-amino]-piperidine-1-carboxylic acid tert-butyl ester (55 mg) in 10 mL of EtOAc and 2 mL of 1M HCl (in ether) were combined and stirred at RT over 2 days. The reaction mixture was decanted, dried and concentrated to give the free base (75 mg) which was converted to the HCl salt with 3 eq. of 1M HCl (in ether) and 5 mL of MeOH. MS m/z 500 (M+1).

### Example 14

Synthesis of 6-[(4-{4-[3-(2-Fluoro-benyloxy)-propoxy]-phenyl}-piperidin-3-ylamino)-methyl]-naphthalene-2-carboxylic acid methyl ester

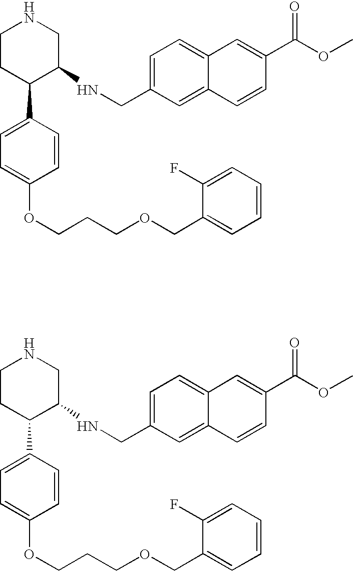

The title compound was prepared as recited in Example 12 utilizing 3-Amino-4-{4-[3-(2-fluoro-benzyloxy)-propoxy]-phenyl}-piperidine-1-carboxylic acid tert-butyl ester, prepared as in Method H. The deprotection step was carried out analogously to that used in Example 13.95% yield. MS m/z 557 (M+1).

### Example 15

Synthesis of 6-[(4-{4-[3-(2-Fluoro-benyloxy)-propoxy]-phenyl}-piperidin-3-ylamino)-methyl]-naphthalene-2-carboxylic acid

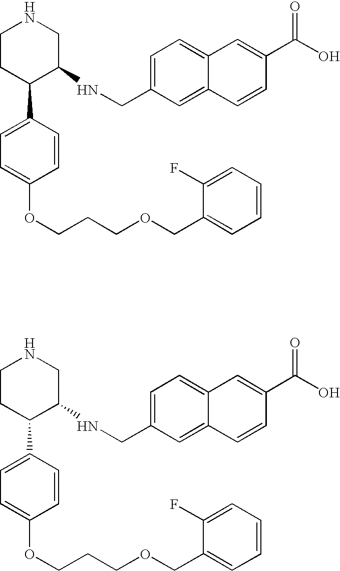

6-[(4-{4-[3-(2-Fluoro-benyloxy)-propoxy]-phenyl}-piperidin-3-ylamino)-methyl]-naphthalene-2-carboxylic acid methyl ester, prepared as in Example 14 (55 mg), was combined with 50 mg of LiOH in 15 mL of MeOH, 5 mL of THF and 2 drops of water and refluxed. The reaction mixture was concentrated and redissolved in 5 mL of EtOAc, a drop of water and 2.5 mL of HCl (1M, in ether) and stirred at RT overnight. The reaction mixture was concentrated, redissolved in EtOAc(10 mL) and 1 mL of sodium bicarbonate(con), separated and dried, to give 35 mg of 6-[(4-{4-[3-(2-fluoro-benyloxy)-propoxy]-phenyl}-piperidin-3-ylamino)-methyl]-naphthalene-2-carboxylic acid, an oil, MS m/z 543 (M+1).

### Example 16

Synthesis of 6-[(4-{4-[3-(2-Fluoro-benzyloxy)-propoxy]-phenyl}-piperidin-3-ylamino)-methyl]-pyridine-2-carboxylic acid methyl ester

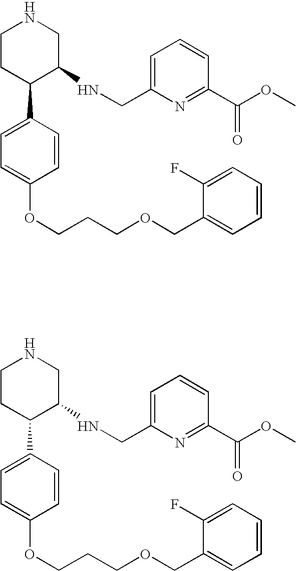

The title compound was prepared as recited in Example 13 utilizing 3-Amino-4-{4-[3-(2-fluoro-benzyloxy)-propoxy]-phenyl}-piperidine-1-carboxylic acid tert-butyl ester, prepared as in Method H, and 6-formyl-pyridine-2-carboxylic acid methyl ester, prepared as in Method I. The deprotection step was carried out analogously to that used in Example 13.90% yield. MS m/z 508 (M+1).

### Example 17

Synthesis of Naphthalene-2-sulfonic acid (4-{4-[3-(2-fluoro-benzyloxy)-propoxy]-phenyl}-piperidin-3-yl)-amide

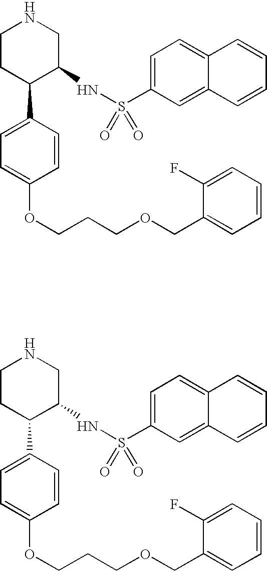

Preparation of 4-{4-[3-(2-Fluoro-benzyloxy)-propoxy]-phenyl}-3-(naphthalene-2-sulfonylamino)-piperidine-1-carboxylic acid tert-butyl ester: 3-Amino-4-{4-[3-(2-fluoro-benzyloxy)-propoxy]-phenyl}-piperidine-1-carboxylic acid tert-butyl ester, prepared as in Method H, (375 mg), pyridine (10 mL), and 2-naphthalene sulfonyl chloride (190 mg) were combined and refluxed for 3 h. The reaction mixture was concentrated and purified by silica gel column, eluted with 5% to 25% EtOAc/hexanes, to afford 4-{4-[3-(2-Fluoro-benzyloxy)-propoxy]-phenyl}-3-(naphthalene-2-sulfonylamino)-piperidine-1-carboxylic acid tert-butyl ester as a white solid (165 mg). The deprotection step was carried out analogously to that used in Example 13. MS m/z 647(M−1)

### Example 18

Synthesis of Naphthalene-2-sulfonic acid (4-{4-[3-(2-fluoro-benzyloxy)-propoxy]-phenyl}-piperidin-3-yl)-amide

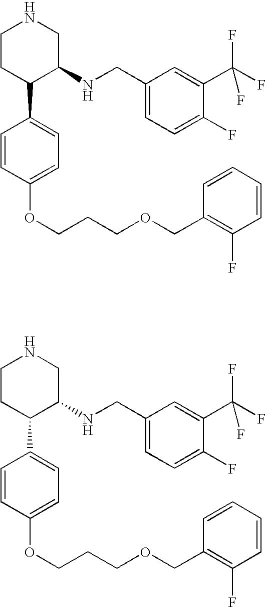

Preparation of 4-{4-[3-(2-Fluoro-benzyloxy)-propoxy]-phenyl}-3-(4-fluoro-3-trifluoromethyl-benzylamino)-piperidine-1-carboxylic acid tert-butyl ester: 3-Amino-4-{4-[3-(2-fluoro-benzyloxy)-propoxy]-phenyl}-piperidine-1-carboxylic acid tert-butyl ester, prepared as in Method H, (200 mg, 0.44 mmol) was dissolved in dichloromethane (15 mL) and 4-fluoro-3-trifluoromethyl benzaldehyde (180 mg, 0.96 mmol), and acetic acid (0.04 mL, 0.44 mmol) were added. The mixture was stirred and sodium triacetoxyborohydride (310 mg, 0.96 mmol) was added and left to stir at RT overnight. The reaction mixture was diluted with dichloromethane (20 mL) and washed with water (25 mL), sat. NaHCO₃ (20 mL), and brine (20 mL). The reaction mixture was dried over MgSO₄, and condensed to afford 4-{4-[3-(2-Fluoro-benzyloxy)-propoxy]-phenyl}-3-(4-fluoro-3-trifluoromethyl-benzylamino)-piperidine-1-carboxylic acid tert-butyl ester, (280 mg, 100%) MS m/z 635 (M+1). The deprotection step was carried out analogously to that used in Example 13 to afford naphthalene-2-sulfonic acid (4-{4-[3-(2-fluoro-benzyloxy)-propoxy]-phenyl}-piperidin-3-yl)-amide. 10% yield, MS m/z 535 (M+1).

### Example 19

Synthesis of {3-[(4-{4-[3-(2-Fluoro-benzyloxy)-propoxy]-phenyl}-piperidin-3-ylamino)-methyl]-phenoxy}-acetic acid methyl ester

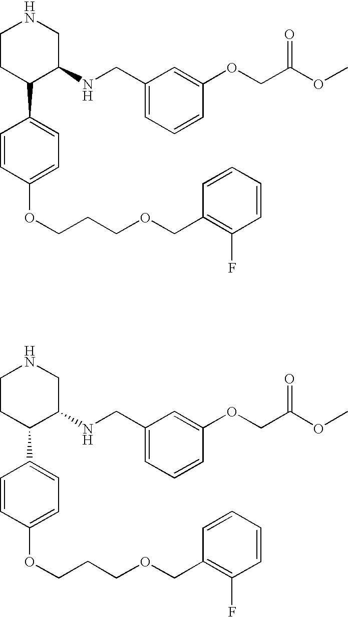

The title compound was prepared analogously to the compound recited in Example 18 utilizing (3-formyl-phenoxy)-acetic acid methyl ester, prepared by reacting 3-hydroxy benzaldehyde (1.0 g, 8.19 mmol) in DMF (25 mL) with K₂CO₃ (2.49 g, 18.01 mmol), sodium iodide (490 mg, 3.28 mmol), and methyl bromoacetate (1.38 g, 9.01 mmol) at overnight at room temperature, instead of 4-fluoro-3-trifluoromethyl benzaldehyde. MS m/z 537 (M+1)

### Example 20

Synthesis of 1-(2-{3-[(4-{4-[3-(2-Fluoro-benzyloxy)-propoxy]-phenyl}-piperidin-3-ylamino)-methyl]-phenoxy}-ethyl)-pyrrolidine-2,5-dione

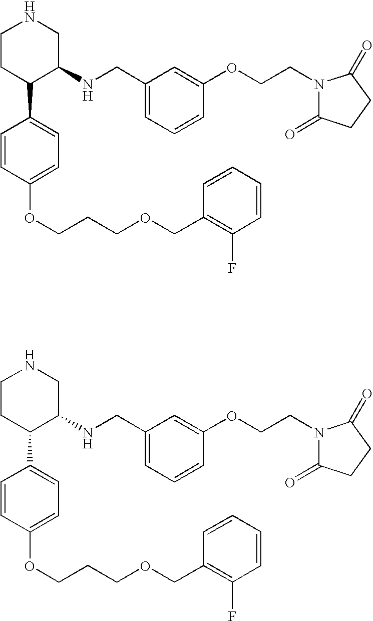

The title compound was prepared analogously to the compound recited in Example 18 utilizing 3-[2-(2,5-dioxo-pyrrolidin-1-yl)-ethoxy]-benzaldehyde, prepared as recited in Method J, instead of 4-fluoro-3-trifluoromethyl benzaldehyde. MS m/z 690 (M+1).

### Example 21

Synthesis of 1-(2-{3-[(4-{4-[3-(2-Fluoro-benzyloxy)-propoxy]-phenyl}-piperidin-3-ylamino)-methyl]-phenoxy}-ethyl)-pyrrolidine-2-one

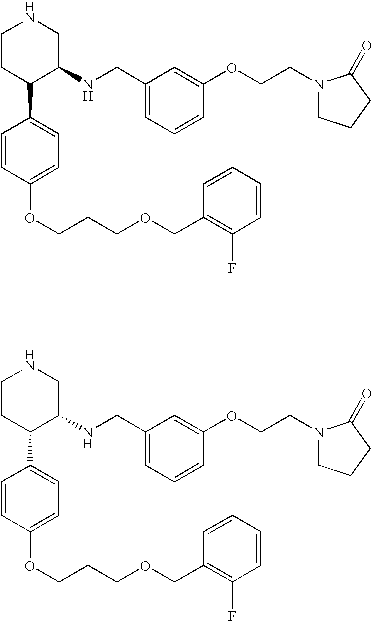

The title compound was prepared analogously to the compound recited in Example 18 utilizing 3-[2-(2-oxo-pyrrolidin-1-yl)-ethoxy]-benzaldehyde, prepared as recited in Method K, instead of 4-fluoro-3-trifluoromethyl benzaldehyde. MS m/z 576 (M+1)

### Example 22

Synthesis of 3-[(1-dimethylcarbamoylmethyl-1, 2,3,4-tetrahydro-quinoline-7-carbonyl)-amino]-4-{4-[3-(2-methoxy-benzyloxy)-propoxy]-phenyl}-piperidine-1-carboxylic acid tert-butyl ester

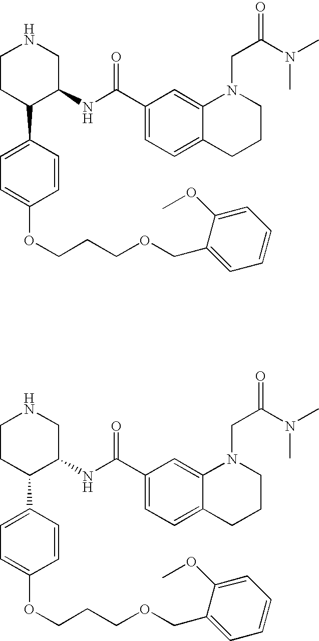

Preparation of 4-{4-[3-(2-methoxy-benzyloxy)-propoxy]-phenyl}-3-[(quinoline-7-carbonyl)-amino]-piperidine-1-carboxylic acid tert-butyl ester: A mixture of 3-amino-4-{4-[3-(2-methoxy-benzyloxy)-propoxy]-phenyl}-piperidine-1-carboxylic acid tert-butyl ester, which can be prepared as recited in Example 9 (0.15 g, 0.32 mmol), quinoline-7-carboxylic acid (0.055 g, 0.32 mmol), HBTU (0.24 g, 0.64 mmol), HOBt (0.086 g, 0.64 mmol) and diisopropylethylamine (0.165 g, 1.28 mmol) in dry DMF (10 mL) was stirred at room temperature overnight and then diluted with ethyl acetate and water. The organic layer was washed with H₂O, brine, dried over Na₂SO₄, and concentrated under vacuum. The residue was subjected to flash column chromatography (50-75% EtOAc/hexanes) to give 0.15 g (75%) of 4-{4-[3-(2-methoxy-benzyloxy)-propoxy]-phenyl}-3-[(quinoline-7-carbonyl)-amino]-piperidine-1-carboxylic acid tert-butyl ester. ¹H NMR (400 MHz, CDCl₃) δ: 8.97 (dd, J=1.5 Hz, J=3.9 Hz, 1H), 8.24 (s, 1H), 8.17 (dd, J=1.1 Hz, 1H), 7.84 (s, 2H), 7.46 (dd, J=4.4 Hz, J=8.3 Hz, 1H), 7.31 (dd, J=2.0 Hz, J=7.3 Hz, 1H), 7.22-7.18 (m, 3H), 6.89 (dd, J=7.3 Hz, 1H), 6.80 (dd, J=7.8 Hz, J=16.1 Hz, 3H), 6.32 (s, 1H), 4.56-4.40 (m, 4H), 4.01 (t, J=6.3 Hz, 2H), 3.74 (s, 3H), 3.64 (t, J=5.9 Hz, 2H), 3.12 (dd, J=1.9 Hz, J=13.6 Hz, 2H), 2.90 (t, J=12.7 Hz, 2H), 2.09-1.96 (m, 4H), 1.44 (s, 9H).

Preparation of 4-{4-[3-(2-methoxy-benzyloxy)-propoxy]-phenyl}-3[(1, 2, 3, 4-tetrahydro-quinoline-7-carbonyl)-amino]-piperidine-1-carboxylic acid tert-butyl ester: To a mixture of 4-{4-[3-(2-methoxy-benzyloxy)-propoxy]-phenyl}-3-[(quinoline-7-carbonyl)-amino]-piperidine-1-carboxylic acid tert-butyl ester (2 g, 3.2 mmol) and NiCl₂.6H₂O (0.76 g, 3.2 mmol) in methanol (20 mL) was added NaBH₄ at 0° C. The reaction mixture was treated with ethyl acetate and water and the organic phase was separated, washed with H₂O and brine, dried over Na₂SO₄, and concentrated under vacuum to give 1.9 g (94%) of 4-{4-[3-(2-methoxy-benzyloxy)-propoxy]-phenyl}-3 [(1, 2, 3, 4-tetrahydro-quinoline-7-carbonyl)-amino]-piperidine-1-carboxylic acid tert-butyl ester. ¹H NMR (400 MHz, CDCl₃) δ: 7.33 (dd, J=2.0 Hz, J=7.8 Hz, 1H), 7.26-7.19 (m, 2H), 7.12 (d, J=8.8 Hz, 2H), 6.90 (dd, J=8.3 Hz, 2H), 6.80 (dd, J=4.9 Hz, J=7.8 Hz, 2H), 6.69-6.67 (m, 2H), 6.01 (s, 1H), 4.54 (s, 2H), 4.51-4.39 (m, 2H), 4.03 (t, J=6.3 Hz, 2H), 3.79 (s, 3H), 3.67 (t, J=5.9 Hz, 2H), 3.27 (t, J=5.9 Hz, 2H), 3.07 (dd, J=4.4 Hz, J=11.7 Hz, 2H), 2.86 (t, J=11.2 Hz, 2H), 2.73 (t, J=6.3 Hz, 2H), 2.07-2.04 (m, 3H), 1.97-1.84 (m, 5H), 1.37 (s, 9H).

Preparation of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester: A mixture of 4-{4-[3-(2-methoxy-benzyloxy)-propoxy]-phenyl}-3[(1, 2, 3, 4-tetrahydro-quinoline-7-carbonyl)-amino]-piperidine-1-carboxylic acid tert-butyl ester (1.0 g, 0.79 mmol), 2-chloro-N,N-dimethylacetamide (0.29 g, 2.4 mmol), Cs₂CO₃ (1.3 g, 4.0 mmol), and KI (0.07 g, 0.4 mmol) in acetonitrile (20 mL) was stirred and refluxed for 24 h. The reaction mixture was filtered, the filtrate concentrated, and the residue was subjected to flash column chromatography (2-5% CH₃OH/CH₂Cl₂) to give 0.9 g (80%) of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester. ¹H NMR (400 MHz, CDCl₃) δ: 7.32 (dd, J=1.5 Hz, J=7.3 Hz, 1H), 7.25-7.14 (m, 2H), 7.13 (d, J=8.8 Hz, 2H), 6.93-6.88 (m, 2H), 6.83-6.64 (m, 3H), 6.63 (dd, J=1.5 Hz, J=7.8 Hz, 1H), 6.07 (d, J=7.3 Hz, 1H), 4.54 (s, 2H), 4.46 (d, J=12.2 Hz, 2H), 4.05-4.00 (m, 4H), 3.76 (s, 3H), 3.59 (t, J=4.4 Hz, 2H), 3.35-3.33 (m, 2H), 3.08-3.04 (m, 2H), 3.02 (s, 3H), 2.93 (s, 3H), 2.89-2.76 (m, 3H), 2.08-2.02 (m, 2H), 1.99-1.88 (m, 4H), 1.76 (s, 1H), 1.37 (s, 9H).

Preparation of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester: To a solution of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester (0.9 g, 1.26 mmol) in 10 mL of dry dioxane was added dropwise an ethereal solution of 2M HCl at 0° C. The resulting solution was stirred at room temperature for 5 h and then treated with a saturated solution of NaHCO₃. The organic layer was separated washed with H₂O and brine, dried over Na₂SO₄, and concentrated under vacuum. The residue was subjected to flash column chromatography (3-5% CH₃OH/CH₂Cl₂) to give 0.4 g (52%) of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester. ¹H NMR (400 MHz, CDCl₃) δ: 7.34 (dd, J=1.5 Hz, J=7.3 Hz, 1H), 7.26-7.20 (m, 3H), 7.15 (d, J=8.8 Hz, 3H), 6.94-6.91 (m, 2H), 6.83-6.76 (m, 5H), 4.54 (s, 2H), 4.46 (d, J=12.2 Hz, 2H), 4.15 (d, J=17.2 Hz, 1H), 4.03-3.96 (m, 2H), 3.76 (s, 3H), 3.66 (t, J=6.4 Hz, 2H), 3.37-3.31 (m, 2H), 3.24-3.16 (m, 2H), 3.02 (s, 3H), 2.94 (s, 3H), 2.81-2.79 (m, 2H), 2.08-2.05 (m, 2H), 2.03-1.97 (m, 2H), 1.80-1.72 (m, 2H).

### Example 23

Synthesis of [1-(2-dimethylamino-ethyl)-1,2,3,4-tetrahydro-quinolin-7-ylmethyl]-(4-{4-[3-(2-methoxybenzyloxy)-propoxy]-phenyl}-piperidin-3-yl)-amine

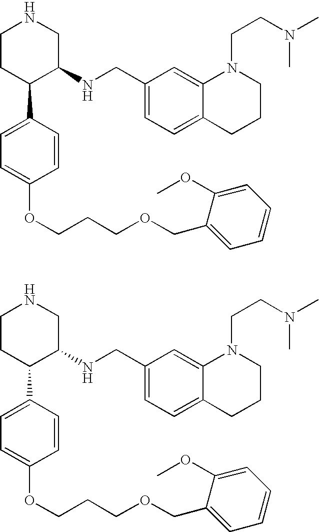

Preparation of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester borane complex: To a solution of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester, prepared as in Example 22 (0.3 g, 0.49 mmol), in 10 mL of THF was added 1M BH₃-dimethylsulfide solution in THF dropwise at 0° C. The resulting mixture was stirred at room temperature overnight and refluxed for 6 h. After removal of solvent, the residue was refluxed in methanol overnight. The methanol was removed under vacuum and the residue diluted in CH₂Cl₂ and washed with saturated solution of NaHCO₃. The organic layer was washed with H₂O and brine, dried over Na₂SO₄, and concentrated under vacuum. The residue was subjected to flash column chromatography (50% EtOAc/hexanes) to give 0.14 g (50%) of 3-[(1-dimethylcarbamoylmethyl-1,2,3,4-tetrahydro-quinoline-7-carbonyl)-amino]-4-(4-[3-(2-methoxy-benzyloxy)-propoxy]-phenyl}-piperidine-1-carboxylic acid tert-butyl ester borane complex. ¹H NMR (400 MHz, CDCl₃) δ: 7.35 (dd, J=1.5 Hz, J=7.3 Hz, 1H), 7.26-7.23 (m, 1H), 7.09-7.05 (m, 2H), 6.94-6.91 (m, 1H), 6.86-6.76 (m, 4H), 6.53 (s, 1H), 6.22 (dd, J=1.0 Hz, J=7.3 Hz, 1H), 4.56 (s, 2H), 4.52-4.36 (m, 1H), 4.08 (t, J=6.3 Hz, 2H), 3.80 (s, 3H), 3.75 (t, J=5.9 Hz, 2H), 3.69-3.57 (m, 2H), 3.43-3.32 (m, 3H), 3.28 (t, J=5.9 Hz, 2H), 3.01-2.95 (m, 2H), 2.90-2.79 (m, 2H), 2.71-2.63 (m, 8H), 2.42-2.37 (m, 1H), 2.15-2.05 (m, 2H), 1.94-1.88 (m, 2H), 1.73 (d, J=12.2 Hz, 2H), 1.59 (br s, 5H).

Preparation of [1-(2-dimethylamino-ethyl)-1,2,3,4-tetrahydro-quinolin-7-ylmethyl]-(4-{4-[3-(2-methoxybenzyloxy)-propoxy]-phenyl}-piperidin-3-yl)-amine: To a solution of 3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester borane complex (0.06 g, 0.1 mmol) in 2 mL of dry dioxane was added an ethereal solution of 2M HCl dropwise at −10° C. The resulting solution was stirred at the same temperature for 4 h and treated with a saturated solution of NaHCO₃ and ethyl acetate at 0° C. The organic layer was washed with H₂O and brine, dried over Na₂SO₄, and concentrated under vacuum. The residue was subjected to flash column chromatography (3-5% CH₃OH/CH₂Cl₂) to give 0.04 g (52%) of [1-(2-dimethylamino-ethyl)-1,2,3,4-tetrahydro-quinolin-7-ylmethyl]-(4-{4-[3-(2-methoxybenzyloxy)-propoxy]-phenyl}-piperidin-3-yl)-amine. ¹H NMR (400 MHz, CDCl₃) δ: 7.35 (dd, J=1.9 Hz, J=7.8 Hz, 1H), 7.27-7.23 (m, 1H), 7.06-7.04 (m, 2H), 6.94-6.91 (m, 1H), 6.87-6.84 (m, 3H), 6.78 (d, J=7.8 Hz, 1H), 6.25 (s, 1H), 6.11 (dd, J=1.5 Hz, J=7.8 Hz, 1H), 4.57 (s, 2H), 4.44-4.36 (m, 1H), 4.09 (t, J=6.3 Hz, 2H), 3.80 (s, 3H), 3.71 (t, J=5.8 Hz, 2H), 3.58 (d, J=12.7, 1H), 3.43-3.40 (m, 2H), 3.37-3.26 (m, 6H), 3.02-2.95 (m, 2H), 2.70-2.63 (m, 4H), 2.42 (t, J=7.3 Hz, 2H), 2.28 (s, 6H), 2.13-2.09 (m, 2H), 1.91-1.88 (m, 2H), 1.75 (d, J=12.2 Hz, 2H).

What is claimed is:

1. A compound of Formula I 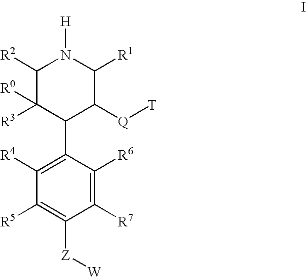
or a pharmaceutically acceptable salt thereof, wherein
R¹ and R² are independently hydrogen or unsubstituted C₁-C₃ alkyl;
R³ is hydrogen, oxo, or thioxo;
R⁰ is hydrogen or unsubstituted C₁-C₃ alkyl provided that when R³ is oxo or thioxo R⁰ is absent;
R⁴, R⁵, R⁶, and R⁷ are independently hydrogen, halogen, carboxyl, substituted or unsubstituted C₁-C₃ alkoxy, or substituted or unsubstituted C₁-C₃ alkyl;
Q is —NR⁸—(CH₂)₀₋₆—, —NR⁹—C(O)—(CH₂)₀₋₆—, wherein 1 to 3 nonadjacent methylene units are replaced with O, NR¹¹, S or a combination thereof;
T is substituted or unsubstituted aryl, substituted or unsubstituted heteroaryl, or substituted or unsubstituted C₁-C₁₂ alkyl
W is absent, substituted or unsubstituted aryl, or substituted or unsubstituted heteroaryl;
Z is —(CH₂)₀₋₆-cycloalkylene-(CH₂)₀₋₆— wherein 0 to 6 nonadjacent methylene units are replaced with O, NR¹², S or a combination thereof, —(CH₂)₀₋₆-heterocycloalkylene-(CH₂)₀₋₆— wherein 0 to 6 nonadjacent methylene units are replaced with O, NR¹², S or a combination thereof, —(CH₂)₀₋₆-arylene-(CH₂)₀₋₆— wherein 0 to 6 nonadjacent methylene units are replaced with O, NR¹², S or a combination thereof, —(CH₂)₀₋₆-heteroarylene-(CH₂)₀₋₆— wherein 0 to 6 nonadjacent methylene units are replaced with O, NR¹², S or a combination thereof, —(CH₂)₀₋₆—C(O)—NR¹¹—(CH₂)₀₋₆— wherein 0 to 6 nonadjacent methylene units are replaced with O, NR¹², S or a combination thereof, —(CH₂)₀₋₆—NR¹¹—C(O)—(CH₂)₀₋₆— wherein 0 to 6 nonadjacent methylene units are replaced with O, NR¹², S or a combination thereof, 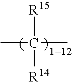
wherein 1 to 6 nonadjacent 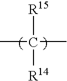
units are replaced with O, NR¹², S or a combination thereof, or
Z, when W is absent, is hydroxyl, substituted or unsubstituted C₁-C₁₂ alkyl wherein 1 to 6 nonadjacent methylene units are replaced with O, NR¹⁶, S or a combination thereof, or —(CH₂)₀₋₆—C(O)—NR¹⁶-(CH₂)₀₋₅—CH₃ wherein 0 to 6 nonadjacent methylene units are replaced with O, NR¹⁶, S or a combination thereof;
R⁸, R⁹ and R¹⁰ are independently hydrogen or substituted or unsubstituted C₁-C₃ alkyl;
R¹¹ and R¹² are independently substituted or unsubstituted C₁-C₃ alkyl; and
R¹⁴ and R¹⁵ are independently hydrogen, substituted or unsubstituted C₁-C₃ alkoxy, substituted or unsubstituted C₁-C₃ alkyl, unsubstituted C₁-C₁₂ alkyl wherein 1 to 6 nonadjacent methylene units are replaced with 0, or R¹⁴ and R¹⁵ together with the carbon to which they are attached form a 3- to 6-membered cycloalkylene or heterocycloalkylene ring; and
R¹⁶ is substituted or unsubstituted C₁-C₃ alkyl or hydrogen.

2. A compound of claim 1, wherein R¹ and R², are hydrogen and R³ is oxo.

3. A compound of claim 1, wherein R⁴, R⁵, R⁶, and R⁷ are independently hydrogen, halogen, carboxyl, C₁-C₃ alkoxy, or C₁-C₃ alkyl.

4. A compound of claim 3, wherein R⁴, R⁵, R⁶, and R⁷ are independently hydrogen, chlorine, fluorine, carboxyl, methoxy or methyl.

5. A compound of claim 1, wherein R⁴, R⁶, and R⁷ are hydrogen and R⁵ is chlorine, fluorine, carboxyl, methoxy or methyl.

6. A compound of claim 1, wherein Q is —NR⁸—(CH₂)₀₋₆—, or —NR⁹—C(O)—(CH₂)₀₋₆— wherein R⁸ and R⁹ are independently unsubstituted C₁-C₃ alkyl.

7. A compound of claim 6, wherein Q is —NH—(CH₂)₀₋₆—, or —NH—C(O)—(CH₂)₀₋₆—.

8. A compound of claim 7, wherein Q is —NH—CH₂—, —NH—CH₂—CH₂—, —NH—CH₂—CH₂—O—CH₂—, or —NH—CH₂—CH₂—O—.

9. A compound of claim 1, wherein T is unsubstituted aryl.

10. A compound of claim 1, wherein T is unsubstituted phenyl, naphthyl, biphenyl, 1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, or 1,2,3,4-tetrahydroindolyl.

11. A compound of claim 10, wherein T is 2-naphthyl, biphen-4-yl, 1,2,3,4-tetrahydroquinolin-6-yl, or 1,2,3,4-tetrahydroquinolin-7-yl.

12. A compound of claim 1, wherein T is substituted aryl

13. A compound of claim 12, wherein T is substituted phenyl, naphthyl, biphenyl, 1,2,3,4-tetrahydroquinolinyl, 2-oxo-1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl.

14. A compound of claim 12, wherein T is phenyl substituted from 1 to 5 times with C₁-C₆alkyl, halo, C₁-C₆alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—NR¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶-(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

15. A compound of claim 14, wherein T is 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, or 4-acetylaminophenyl.

16. A compound of claim 1, wherein T is biphenyl substituted from 1 to 9 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C_-C₆ alkyl)₀₋₁-, HO—C(O)—(C₁-C₆ alkyl) o—, (C₁-C₆ alkyl)-C(O)—(C₁-C₆ alkyl) o—, (C₁-C₆ alkyl)-S(O)₂—NR¹⁶—(C₁-C₆ alkyl)₀₋₁-, (C₁-C₆ alkyl)-NR¹-S(O)₂—(C₁-C₆ alkyl)₀₋₁-, or HO—(C₁-C₆ alkyl), wherein each R¹⁶ is independently H or C₁-C₆ alkyl or a combination thereof.

17. A compound of claim 1, wherein T is naphthyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 3,4-dihydro-2H-benzo[1,4]oxazinyl, 3-oxo-3,4-dihydro-2H-benzo[1,4]oxazinyl, 2,3-dihydroindolyl, or 1,2,3,4-tetrahydroindolyl substituted from 1 to 7 times with, C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)_, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

18. A compound of claim 17, wherein T is 6-methoxy-2-naphthyl, 7-methoxy-2-naphthyl, 6-methyl-2-naphthyl, 6-hydroxy-2-naphthyl, 7-methyl-2-naphthyl, 6-trifluoromethyl-2-naphthyl, 7-trifluoromethyl-2-naphthyl, 6-fluoro-2-naphthyl, 7-fluoro-2-naphthyl, 6-chloro-2-naphthyl, 7-chloro-2-naphthyl, 6-(2-acetoxy-ethyl)-2-naphthyl, 7-(2-acetoxy-ethyl)-2-naphthyl, 1-(3-hydroxypropyl)-3,4-dihydro-2H-quinolin-7-yl, 1-acetyl-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, or 1-(2-acetoxy-ethyl)-3,4-dihydro-2H-quinolin-7-yl.

19. A compound of claim 1, wherein T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

20. A compound of claim 1, wherein T is unsubstituted heteroaryl.

21. A compound of claim 20, wherein T is quinolinyl, indolyl, benzofuryl, isoquinolinyl, pyridyl, pyrimidinyl, pyrazinyl, or quinoxalinyl.

22. A compound of claim 21, wherein T is 2-quinolinyl, 6-quinolinyl, 7-quinolinyl, 6-isoquinolinyl, 2-pyridyl, 5-benzofuryl, 2-pyrimidinyl, 2-pyrazinyl, or 2-quinoxalinyl.

23. A compound of claim 1, wherein T is substituted heteroaryl.

24. A compound of claim 23, wherein T is substituted quinolinyl, indolyl, benzofuryl, isoquinolinyl, pyridyl, pyrimidinyl, pyrazinyl, or quinoxalinyl.

25. A compound of claim 24, wherein T is quinolinyl, isoquinolinyl or quinoxalinyl substituted from 1 to 7 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶-(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

26. A compound of claim 24, wherein T is pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁, (C₁-C₆ alkyl)-NR¹⁶-S(O)₂—(C₁-C₆ alkyl)₀₋₁-, or HO—(C₁-C₆ alkyl), wherein each R¹⁶ is independently H or C₁-C₆ alkyl or a combination thereof.

27. A compound of claim 1, wherein T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl.

28. A compound of claim 27, wherein the N-substituent is C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶-(C₁-C₆ alkyl) ol-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl.

29. A compound of claim 1, wherein Z is 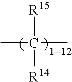
wherein 1 to 6 nonadjacent 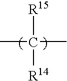
units are replaced with 0.

30. A compound of claim 1, wherein R¹⁴ and R¹⁵ are hydrogen.

31. A compound of claim 1, wherein Z is
—(CH₂)₀₋₆—C(O)—NR¹¹—(CH₂)₀₋₆— wherein 0 to 6 nonadjacent methylene units are replaced with O, NR¹², S or a combination thereof; or
—(CH₂)₀₋₆—NR¹¹—(C(O)—CH₂)₀₋₆— wherein 0 to 6 nonadjacent methylene units are replaced with O, NR¹², S or a combination thereof; and
R¹¹ and R¹² are as defined in claim 1.

32. A compound of claim 29, wherein Z is —O—(CH₂)₂₋₃—O—(CH₂)₁₋₂—, —O—(CH₂)₃₋₄—O—, O—(CH₂)₁₋₂-, —(CH₂)—O—(CH₂)₂₋₃—O—(CH₂)₀₋₁-, —C(O)—NR¹¹—(CH₂)₂—, —C(O)—NR¹¹—(CH₂)₂—O—, or —O—(CH₂)₃—S—(CH₂)₁—; and
R¹¹ is as defined in claim 1.

33. A compound of claim 1, wherein when W is absent, Z is hydroxyl, C₁-C₁₂ alkyl wherein 1 to 6 nonadjacent methylene units are replaced with O, or —(CH₂)₀₋₆—C(O)—NR¹⁶—(CH₂)₀₋₅—CH₃ wherein 0 to 6 nonadjacent methylene units are replaced with O; and
R¹⁶ is as defined in claim 1.

34. A compound of claim 1, wherein Z is —O—(CH₂)₃—O—(CH₂)—.

35. A compound of claim 1, wherein W is unsubstituted or substituted phenyl.

36. A compound of claim 1, wherein W is 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, or 4-acetylaminophenyl.

37. A compound of claim 1, wherein W is 2-methoxyphenyl.

38. A compound of claim 1, wherein W is unsubstituted or substituted heteroaryl

39. A compound of claim 38, wherein W is unsubstituted indolyl.

40. A compound of claim 39, wherein W is 1H-Indol-3-yl.

41. A compound of claim 1, wherein Z is —O—(CH₂)₃—O—CH₂—, and W is 2-methoxyphenyl.

42. A compound of claim 1, wherein Q is —NH—CH₂— or —NR⁸—CH₂—; T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl; and R⁸ is C₁-C₃ alkyl.

43. A compound of claim 1 having the formula II or III 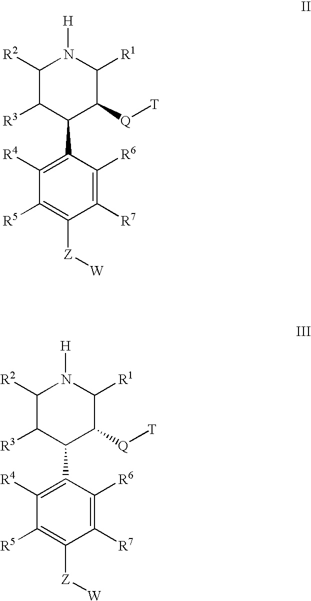
or a pharmaceutically acceptable salt thereof, wherein
R¹, R², R³, R⁴, R⁵, R⁶, R⁷, R⁸, R⁹, R¹⁰, R¹¹, R¹², R¹⁴, R¹⁵, R¹⁶, Q, T, Z, and W are as defined above in claim 1.

44. A compound of Formula IV or V 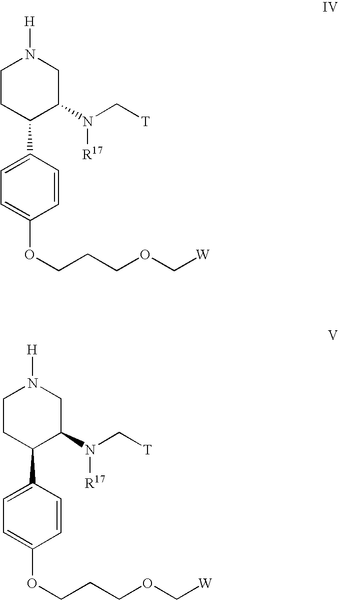
or a pharmaceutically acceptable salt thereof, wherein
T is substituted or unsubstituted aryl, or substituted or unsubstituted heteroaryl;
W is substituted or unsubstituted aryl, or substituted or unsubstituted heteroaryl; and
R¹⁷ is hydrogen or C₁-C₃ alkyl.

45. A compound of claim 44, wherein T is substituted aryl.

46. A compound of claim 45, wherein T is substituted phenyl, naphthyl, biphenyl, 1,2,3,4-tetrahydroquinolinyl, 2-oxo-1,2,3,4-tetrahydroquinolinyl, 1,2,3,4-tetrahydro-naphthyl, 1,2,3,4-tetrahydroisoquinolinyl, 1,2,3,4-tetrahydroquinoxalinyl, 1,2,3,4-tetrahydroindolyl, 2,3-dihydroindolyl, 3-oxo-3,4-dihydro-2H-benzo[1,4]oxazinyl, or 3,4-dihydro-2H-benzo[1,4]oxazinyl.

47. A compound of claim 44, wherein T is naphthyl, 1,2,3,4-tetrahydroquinolinyl, 2-oxo-1,2,3,4-tetrahydroquinolinyl, 1,2,3,4-tetrahydronaphthyl, 1,2,3,4-tetrahydroisoquinolinyl, 1,2,3,4-tetrahydroquinoxalinyl, 3,4-dihydro-2H-benzo[1,4]oxazinyl, 3-oxo-3,4-dihydro-2H-benzo[1,4]oxazinyl, 2,3-dihydroindolyl, or 1,2,3,4-tetrahydroindolyl substituted from 1 to 7 times with, C₁-C₆ alkyl, halo, hydroxy, oxo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

48. A compound of claim 44, wherein T is unsubstituted naphthyl, unsubstituted 4-trifluoromethylphenyl, unsubstituted 1,2,3,4-tetrahydroquinolin-7-yl, 1-(2-ethoxy-2-oxoethyl)-5-indolyl, 1-(2-acetylaminoethyl)-5-indolyl, 1-(3-methoxypropyl)-5-indolyl, 1-acetamidyl-5-indolyl, 1-(2-acetoxyethyl)-5-indolyl, 1-(3-methoxy-3-oxopropyl)-5-indolyl, 1-(2-methoxy-2-oxoethyl)-5-indolyl, 1-(2-ethoxy-2-oxoethyl)-6-indolyl, 1-(2-acetylaminoethyl)-6-indolyl, 1-(3-methoxypropyl)-6-indolyl, 1-acetamidyl-6-indolyl, 1-(2-acetoxyethyl)-6-indolyl, 1-(3-methoxy-3-oxopropyl)-6-indolyl, 1-(2-methoxy-2-oxoethyl)-6-indolyl, 4-(2-ethoxy-2-oxoethyl)-3-oxo-3,4-dihydro-2H-benzo[1,4]oxazin-6-yl, 3-oxo-3,4-dihydro-2H-benzo[1,4]oxazin-6-yl, 4-(3-methoxypropyl)-3-oxo-3,4-dihydro-2H-benzo[1,4]oxazin-6-yl, 4-(2-acetylaminoethyl)-3-oxo-3,4-dihydro-2H-benzo[1,4]oxazin-6-yl, 4-acetamidyl-3-oxo-3,4-dihydro-2H-benzo[1,4]oxazin-6-yl, 4-(2-acetoxyethyl)-3-oxo-3,4-dihydro-2H-benzo[1,4]oxazin-6-yl, 4-(3-methoxy-3-oxopropyl)-3-oxo-3,4-dihydro-2H-benzo[1,4]oxazin-6-yl, 4-(2-methoxy-2-oxoethyl)-3-oxo-3,4-dihydro-2H-benzo[1,4]oxazin-6-yl, 1-(3-hydroxypropyl)-3,4-dihydro-2H-quinolin-7-yl, 1-(3-hydroxypropyl)-2-oxo-3,4-dihydro-2H-quinolin-7-yl, 1-acetyl-3,4-dihydro-2H-quinolin-6-yl, 1-acetyl-2-oxo-3,4-dihydro-2H-quinolin-6-yl, 1-(4-thiazolylmethyl)-3,4-dihydro-2H-quinolin-7-yl, 1-acetamidyl-3,4-dihydro-2H-quinolin-7-yl, 1-acetamidyl-2-oxo-3,4-dihydro-2H-quinolin-7-yl, 1-acetamidyl-3,4-dihydro-2H-quinolin-6-yl, 1-acetamidyl-2-oxo-3,4-dihydro-2H-quinolin-6-yl, 1-(2-acetylaminoethyl)-3,4-dihydro-2H-quinolin-7-yl, 1-(3-methoxy-3-oxopropyl)-3,4-dihydro-2H-quinolin-7-yl, 1-(3-methoxypropyl)-3,4-dihydro-2H-quinolin-7-yl, 1-(2-methoxy-2-oxoethyl)-3,4-dihydro-2H-quinolin-7-yl, 1-(2-ethoxy-2-oxoethyl)-3,4-dihydro-2H-quinolin-7-yl, 1-(2-acetylaminoethyl)-3,4-dihydro-2H-quinolin-6-yl, 1-(3-methoxy-3-oxopropyl)-3,4-dihydro-2H-quinolin-6-yl, 1-(3-methoxypropyl)-3,4-dihydro-2H-quinolin-6-yl, 1-(2-methoxy-2-oxoethyl)-3,4-dihydro-2H-quinolin-6-yl, 1-(2-ethoxy-2-oxoethyl)-3,4-dihydro-2H-quinolin-6-yl, 2-oxo-1,2,3,4-tetrahydro-2H-quinolin-7-yl, 2-oxo-1,2,3,4-tetrahydro-2H-quinolin-6-yl, 1-(2-acetylaminoethyl)-2-oxo-3,4-dihydro-2H-quinolin-7-yl, 1-(3-methoxy-3-oxopropyl)-2-oxo-3,4-dihydro-2H-quinolin-7-yl, 1-(3-methoxypropyl)-2-oxo-3,4-dihydro-2H-quinolin-7-yl, 1-(2-methoxy-2-oxoethyl)-2-oxo-3,4-dihydro-2H-quinolin-7-yl, 1-(2-ethoxy-2-oxoethyl)-2-oxo-3,4-dihydro-2H-quinolin-7-yl, 1-(2-acetylaminoethyl)-2-oxo-3,4-dihydro-2H-quinolin-6-yl, 1-(3-methoxy-3-oxopropyl)-2-oxo-3,4-dihydro-2H-quinolin-6-yl, 1-(3-methoxypropyl)-2-oxo-3,4-dihydro-2H-quinolin-6-yl, 1-(2-methoxy-2-oxoethyl)-2-oxo-3,4-dihydro-2H-quinolin-6-yl, 1-(2-ethoxy-2-oxoethyl)-2-oxo-3,4-dihydro-2H-quinolin-6-yl, 1-(2-acetoxyethyl)-2-oxo-3,4-dihydro-2H-quinolin-6-yl, 1-(2-acetoxyethyl)-2-oxo-3,4-dihydro-2H-quinolin-7-yl, 1-(2-acetoxyethyl)-3,4-dihydro-2H-quinolin-6-yl or 1-(2-acetoxyethyl)-3,4-dihydro-2H-quinolin-7-yl.

49. A compound of claim 44, wherein T is quinolinyl, isoquinolinyl or quinoxalinyl substituted from 1 to 7 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶-(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁-C₆ alkyl), wherein each R¹⁶ is independently H or C₁-C₆ alkyl or a combination thereof.

50. A compound of claim 44, wherein T is pyridyl, indolyl, pyrimidinyl, or pyrazinyl, substituted from 1 to 5 times with C₁-C₆ alkyl, halo, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶—(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁, (C₁-C₆ alkyl)-S(O)₂—NR¹⁶—(C₁-C₆ alkyl)₀₋₁-, (C₁-C₆ alkyl)-NR¹⁶—S(O)₂—(C₁-C₆ alkyl)₀₋₁-, or HO—(C₁C₆ alkyl), wherein each R¹⁶ is independently H or C₁-C₆ alkyl or a combination thereof.

51. A compound of claim 44, wherein T is N-substituted 1,2,3,4-tetrahydroquinolin-7-yl, N-substituted 1,2,3,4-tetrahydroquinolin-6-yl, N-substituted 2-oxo-1,2,3,4-tetrahydroquinolin-7-yl, N-substituted 2-oxo-1,2,3,4-tetrahydroquinolin-6-yl, N-substituted 3-oxo-3,4-dihydro-2H-benzo[1,4]oxazin-6-yl, N-substituted 3-oxo-3,4-dihydro-2H-benzo[1,4]oxazin-7-yl, N-substituted 2-oxo-4a,8a-dihydro-2H-chromen-7-yl, N-substituted 2,3-dihydroindol-6-yl, N-substituted 2-oxo-2,3-dihydroindol-6-yl, N-substituted 2,3-dihydroindol-5-yl, N-substituted 2-oxo-2,3-dihydroindol-5-yl, N-substituted 6-indolyl or N-substituted 5-indolyl.

52. A compound of claim 51, wherein the N-substituent is C₁-C₆ alkyl, C₁-C₆ alkyl wherein 1 to 3 nonadjacent carbons are replaced with O, NR¹⁶, S or a combination thereof, (C₁-C₆ alkyl)-C(O)—O—(C₁-C₆ alkyl)₀₋₁-, (C₁-C₆ alkyl)-O—C(O)—(C₁-C₆ alkyl)₀₋₁-, (C₁-C₆ alkyl)-C(O)—N(R¹⁶)—, (C₁-C₆ alkyl)-NR¹⁶—C(O)—(C₁-C₆ alkyl)₀₋₁-, trifluoromethyl, (C₁-C₆ alkyl)-C(O)—NR¹⁶-(C₁-C₆ alkyl)₀₋₁-, HO—C(O)—(C₁-C₆ alkyl)₀₋₁-, (C₁-C₆ alkyl)-C(O)—(C₁-C₆ alkyl)₀₋₁-, (C₁-C₆ alkyl)-S(O)₂—NR¹⁶—(C₁-C₆ alkyl)₀₋₁-, (C₁-C₆ alkyl)-NR¹⁶-S(O)₂—(C₁-C₆ alkyl)-, or HO—(C₁-C₆ alkyl), wherein each R¹⁶ is independently H or C₁-C₆ alkyl.

53. A compound of claim 44, wherein W is unsubstituted or substituted phenyl.

54. A compound of claim 53, wherein W is 2-trifluoromethylphenyl, 3-trifluoromethylphenyl, 4-trifluoromethylphenyl, 2-chlorophenyl, 3-chlorophenyl, 4-chlorophenyl, 3,4-dichlorophenyl, 3,5-dichlorophenyl, 2-fluorophenyl, 3-fluorophenyl, 4-fluorophenyl, 3,4-difluorophenyl, 3,5-difluorophenyl, 2-methoxyphenyl, 3-methoxyphenyl, 4-methoxyphenyl, 3,4-dimethoxyphenyl, 3,5-dimethoxyphenyl, 2-methylphenyl, 3-methylphenyl, 4-methylphenyl, 3,4-dimethylphenyl, 3,5-dimethylphenyl, 2-chloro-4-fluorophenyl, 4-fluoro-2-trifluoromethylphenyl, 2-(2-acetoxy-ethyl)-phenyl, 3-(2-acetoxy-ethyl)-phenyl, 4-(2-acetoxy-ethyl)-phenyl, N,N-dimethyl-benzamide-4-yl, or 4-acetylaminophenyl.

55. A compound of claim 44, wherein W is 2-methoxyphenyl.

56. A compound of claim 44, wherein T is unsubstituted naphthyl, unsubstituted 4-trifluoromethylphenyl, unsubstituted 1,2,3,4-tetrahydroquinolin-7-yl, 1-(3-hydroxypropyl)-3,4-dihydro-2H-quinolin-7-yl, or 1-(2-acetoxy-ethyl)-3,4-dihydro-2H-quinolin-7-yl and W is 2-methoxyphenyl.

57. The compound
(4-{4-[3-(2-methoxy-benzyloxy)-propoxy]-phenyl}-piperidin-3-yl)-naphthalen-2-ylmethyl-amine,
(4-{4-[3-(2-methoxy-benzyloxy)-propoxy]-phenyl}-piperidin-3-yl)-(6-methoxy-naphthalen-2-ylmethyl)-amine,
(4-{4-[3-(2-methoxy-benzyloxy)-propoxy]-phenyl}-piperidin-3-yl)-quinolin-7-ylmethyl-amine,
(4-{4-[3-(2-methoxy-benzyloxy)-propoxy]-phenyl}-piperidin-3-yl)-(1,2,3,4-tetrahydro-quinolin-7-ylmethyl)-amine,
(4-{4-[3-(2-methoxy-benzyloxy)-propoxy]-phenyl}-piperidin-3-yl)-methyl-naphthalen-2-ylmethyl-amine,
6-[(4-{4-[3-(2-methoxy-benzyloxy)-propoxy]-phenyl}-piperidin-3-ylamino)-methyl]-naphthalen-2-ol,
benzofuran-5-ylmethyl-(4-{4-[3-(2-methoxy-benzyloxy)-propoxy]-phenyl}-piperidin-3-yl)-amine,
(1H-indol-5-ylmethyl)-(4-{4-[3-(2-methoxy-benzyloxy)-propoxy]-phenyl}-piperidin-3-yl)-amine;
6-[(4-[3-(2-methoxy-benzyloxy)-propoxyl]-phenyl}-piperidin-3-ylamino)-methyl]-naphthalene-1-carboxylic acid methyl ester;
6-[(4-[4-(2-methoxy-benzyloxy)-propoxyl]-phenyl}-piperidin-3-ylamino)-methyl]-naphthalene-1-carboxylic acid;
naphthalene-1-carboxylic acid (4-{4-[3-(2-methoxy-benzyloxy)-propoxy]-phenyl}-piperidin-3-yl)-amide;
6-[(4-{4-[3-(2-methoxy-benyloxy)-propoxy]-phenyl}-piperidin-3-ylamino)-methyl]-naphthalene-2-carboxylic acid methyl ester;
(4-{4-[3-(2-fluoro-benzyloxy)-propoxy]-phenyl}-piperidin-3-yl)-quinolin-7-ylmethyl-amine;
6-[(4-{4-[3-(2-fluoro-benyloxy)-propoxy]-phenyl}-piperidin-3-ylamino)-methyl]-naphthalene-2-carboxylic acid methyl ester;
6-[(4-{4-[3-(2-fluoro-benyloxy)-propoxy]-phenyl}-piperidin-3-ylamino)-methyl]-naphthalene-2-carboxylic acid;
6-[(4-{4-[3-(2-fluoro-benzyloxy)-propoxy]-phenyl}-piperidin-3-ylamino)-methyl]-pyridine-2-carboxylic acid methyl ester;
naphthalene-2-sulfonic acid (4-{4-[3-(2-fluoro-benzyloxy)-propoxy]-phenyl}-piperidin-3-yl)-amide;
(4-{4-[3-(2-fluoro-benzyloxy)-propoxy]-phenyl}-piperidin-3-yl)-(4-fluoro-3-trifluoromethyl-benzyl)-amine;
{3-[(4-{4-[3-(2-fluoro-benzyloxy)-propoxy]-phenyl}-piperidin-3-ylamino)-methyl]-phenoxy}-acetic acid methyl ester;
1-(2-{3-[(4-{4-[3-(2-fluoro-benzyloxy)-propoxy]-phenyl}-piperidin-3-ylamino)-methyl]-phenoxy}-ethyl)-pyrrolidine-2,5-dione;
1-(2-{3-[(4-{4-[3-(2-fluoro-benzyloxy)-propoxy]-phenyl}-piperidin-3-ylamino)-methyl]-phenoxy}-ethyl)-pyrrolidine-2-one;
3-[(1-dimethylcarbamoylmethyl-1, 2, 3, 4-tetrahydro-quinoline-7-carbonyl)-amino]-4-{4-[3-(2-methoxy-benzyloxy)-propoxy]-phenyl}-piperidine-1-carboxylic acid tert-butyl ester; or
[1-(2-dimethylamino-ethyl)-1,2,3,4-tetrahydro-quinolin-7-ylmethyl]-(4-{4-[3-(2-methoxybenzyloxy)-propoxy]-phenyl}-piperidin-3-yl)-amine.

58. A pharmaceutical composition comprising a compound of any of claims 1-57, admixed with a pharmaceutically acceptable carrier, diluent, or excipient.

59. A method of inhibiting renin in a mammal comprising administering to the mammal in need thereof an effective amount of a compound of any of claims 1-57.

60. A method of treating or preventing hypertension in a mammal comprising administering to the mammal in need thereof an effective amount of a compound of any of claims 1-57.

61. A method of treating or preventing congestive heart failure in a mammal comprising administering to the mammal in need thereof an effective amount of a compound of any of claims 1-57.

62. A method of treating or preventing stroke in a mammal comprising administering to the mammal in need thereof an effective amount of a compound of any of claims 1-57.

63. A method of treating or preventing myocardial infarction in a mammal comprising administering to the mammal in need thereof an effective amount of a compound of any of claims 1-57.

64. A method of treating or preventing glaucoma in a mammal comprising administering to the mammal in need thereof an effective amount of a compound of any of claims 1-57.

65. A method of providing end organ protection in a mammal comprising administering to the mammal in need thereof an effective amount of a compound of any of claims 1-57.

66. A method of treating or preventing hyperaldosteronism in a mammal comprising administering to the mammal in need thereof an effective amount of a compound of any of claims 1-57.

67. A process for preparing a compound of claim 1 comprising the steps of:
a) alkylation of piperidine 1 to afford the intermediate 2 wherein R²⁰ along with the oxygen to which it is attached, is equivalent to -Z-W as defined in claim 1; 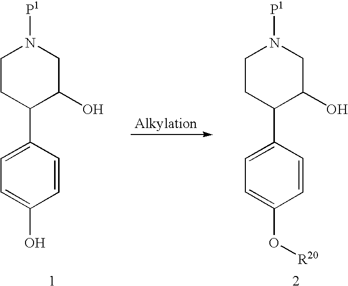
b) oxidation of 2 to afford the piperidinone intermediate 3; 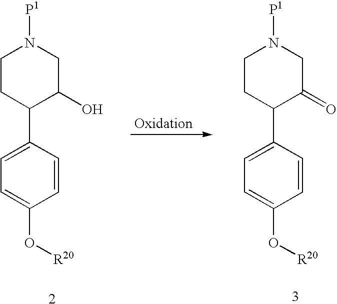
c) contacting 3 with a suitable amine to afford the intermediate 4, wherein R²¹, along with the nitrogen to which it is attached is equivalent to -Q-T as defined in claim 1; 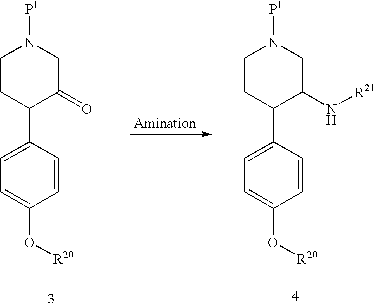
d) deprotection of 4 to afford 5 [chemical structure]
